SEQUENCE LISTING (24 sequences)

SEQ ID NO: 1 - protein, 48 aa, Homo sapiens
NPICKGCLSCSKDNGCSRCQQKLFFFLRREGMRQYGECLHSCPSGYYG

SEQ ID NO: 2 - protein, 123 aa, Homo sapiens
QGNRWRRSKRASYVSNPICKGCLSCSKDNGCSRCQQKLFFFLRREGMRQYGECLHSCPSGYYGHRAPDMNRCARCRIENCDSCFSKDACTKCKVGFYLHRGRCFDECPDGFAPLEETMECVEG

SEQ ID NO: 3 - protein, 118 aa, synthetic construct
NPPTFSPALLVVTEGDNATFTCSFSNTSESFHVVWHRESPSGQTDTLAAFPEDRSQPGQDCRFRVTQLPNGRDFHMSVVRARRNDSGTYVCGVISLAPKIQIKESLRAELRVTERRAE

SEQ ID NO: 4 - protein, 118 aa, Homo sapiens
NPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAE

SEQ ID NO: 5 - protein, 69 aa, synthetic construct
HVVWHRESPSGQTDTLAAFPEDRSQPGQDCRFRVTQLPNGRDFHMSVVRARRNDSGTYVCGVISLAPKI

SEQ ID NO: 6 - protein, 10 aa, synthetic construct
GSGSGGSGSG

SEQ ID NO: 7 - protein, 261 aa, synthetic construct
QGNRWRRSKRASYVSNPICKGCLSCSKDNGCSRCQQKLFFFLRREGMRQYGECLHSCPSGYYGHRAPDMNRCARCRIENCDSCFSKDACTKCKVGFYLHRGRCFDECPDGFAPLEETMECVEGGSGSGGSGSGNPPTFSPALLVVTEGDNATFTCSFSNTSESFHVVWHRESPSGQTDTLAAFPEDRSQPGQDCRFRVTQLPNGRDFHMSVVRARRNDSGTYVCGVISLAPKIQIKESLRAELRVTERRAERSDYKDDDDK

SEQ ID NO: 8 - protein, 21 aa, synthetic construct
MQFRLFSFALIILNCMDYSHC

SEQ ID NO: 9 - protein, 282 aa, synthetic construct
MQFRLFSFALIILNCMDYSHCQGNRWRRSKRASYVSNPICKGCLSCSKDNGCSRCQQKLFFFLRREGMRQYGECLHSCPSGYYGHRAPDMNRCARCRIENCDSCFSKDACTKCKVGFYLHRGRCFDECPDGFAPLEETMECVEGGSGSGGSGSGNPPTFSPALLVVTEGDNATFTCSFSNTSESFHVVWHRESPSGQTDTLAAFPEDRSQPGQDCRFRVTQLPNGRDFHMSVVRARRNDSGTYVCGVISLAPKIQIKESLRAELRVTERRAERSDYKDDDDK

SEQ ID NO: 10 - protein, 936 aa, Homo sapiens
MRPRSGGRPGATGRRRRRLRRRPRGLRCSRLPPPPPLPLLLGLLLAAAGPGAARAKETAFVEVVLFESSPSGDYTTYTTGLTGRFSRAGATLSAEGEIVQMHPLGLCNNNDEEDLYEYGWVGVVKLEQPELDPKPCLTVLGKAKRAVQRGATAVIFDVSENPEAIDQLNQGSEDPLKRPVVYVKGADAIKLMNIVNKQKVARARIQHRPPRQPTEYFDMGIFLAFFVVVSLVCLILLVKIKLKQRRSQNSMNRLAVQALEKMETRKFNSKSKGRREGSCGALDTLSSSSTSDCAICLEKYIDGEELRVIPCTHRFHRKCVDPWLLQHHTCPHCRHNIIEQKGNPSAVCVETSNLSRGRQQRVTLPVHYPGRVHRTNAIPAYPTRTSMDSHGNPVTLLTMDRHGEQSLYSPQTPAYIRSYPPLHLDHSLAAHRCGLEHRAYSPAHPFRRPKLSGRSFSKAACFSQYETMYQHYYFQGLSYPEQEGQSPPSLAPRGPARAFPPSGSGSLLFPTVVHVAPPSHLESGSTSSFSCYHGHRSVCSGYLADCPGSDSSSSSSSGQCHCSSSDSVVDCTEVSNQGVYGSCSTFRSSLSSDYDPFIYRSRSPCRASEAGGSGSSGRGPALCFEGSPPPEELPAVHSHGAGRGEPWPGPASPSGDQVSTCSLEMNYSSNSSLEHRGPNSSTSEVGLEASPGAAPDLRRTWKGGHELPSCACCCEPQPSPAGPSAGAAGSSTLFLGPHLYEGSGPAGGEPQSGSSQGLYGLHPDHLPRTDGVKYEGLPCCFYEEKQVARGGGGGSGCYTEDYSVSVQYTLTEEPPPGCYPGARDLSQRIPIIPEDVDCDLGLPSDCQGTHSLGSWGGTRGPDTPRPHRGLGATREEERALCCQARALLRPGCPPEEAGAVRANFPSALQDTQESSTTATEAAGPRSHSADSSSPGA

SEQ ID NO: 11 - protein, 783 aa, Homo sapiens
MSGGHQLQLAALWPWLLMATLQAGFGRTGLVLAAAVESERSAEQKAIIRVIPLKMDPTGKLNLTLEGVFAGVAEITPAEGKLMQSHPLYLCNASDDDNLEPGFISIVKLESPRRAPRPCLSLASKARMAGERGASAVLFDITEDRAAAEQLQQPLGLTWPVVLIWGNDAEKLMEFVYKNQKAHVRIELKEPPAWPDYDVWILMTVVGTIFVIILASVLRIRCRPRHSRPDPLQQRTAWAISQLATRRYQASCRQARGEWPDSGSSCSSAPVCAICLEEFSEGQELRVISCLHEFHRNCVDPWLHQHRTCPLCMFNITEGDSFSQSLGPSRSYQEPGRRLHLIRQHPGHAHYHLPAAYLLGPSRSAVARPPRPGPFLPSQEPGMGPRHHRFPRAAHPRAPGEQQRLAGAQHPYAQGWGLSHLQSTSQHPAACPVPLRRARPPDSSGSGESYCTERSGYLADGPASDSSSGPCHGSSSDSVVNCTDISLQGVHGSSSTFCSSLSSDFDPLVYCSPKGDPQRVDMQPSVTSRPRSLDSVVPTGETQVSSHVHYHRHRHHHYKKRFQWHGRKPGPETGVPQSRPPIPRTQPQPEPPSPDQQVTRSNSAAPSGRLSNPQCPRALPEPAPGPVDASSICPSTSSLFNLQKSSLSARHPQRKRRGGPSEPTPGSRPQDATVHPACQIFPHYTPSVAYPWSPEAHPLICGPPGLDKRLLPETPGPCYSNSQPVWLCLTPRQPLEPHPPGEGPSEWSSDTAEGRPCPYPHCQVLSAQPGSEEELEELCEQAV

SEQ ID NO: 12 - protein, 243 aa, Homo sapiens
MQFRLFSFALIILNCMDYSHCQGNRWRRSKRASYVSNPICKGCLSCSKDNGCSRCQQKLFFFLRREGMRQYGECLHSCPSGYYGHRAPDMNRCARCRIENCDSCFSKDFCTKCKVGFYLHRGRCFDECPDGFAPLEETMECVEGCEVGHWSEWGTCSRNNRTCGFKWGLETRTRQIVKKPVKDTILCPTIAESRRCKMTMRHCPGGKRTPKAKEKRNKKKKRKLIERAQEQHSVFLATDRANQ

SEQ ID NO: 13 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 14 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 15 - DNA, 19 bp, synthetic construct
  source 1..19: mol_type = other DNA
ccacacccttctccaatcc

SEQ ID NO: 16 - DNA, 20 bp, synthetic construct
  source 1..20: mol_type = other DNA
tgccagtttctttggctctt

SEQ ID NO: 17 - DNA, 21 bp, synthetic construct
  source 1..21: mol_type = other DNA
cctactggcatttgctgaacg

SEQ ID NO: 18 - DNA, 21 bp, synthetic construct
  source 1..21: mol_type = other DNA
agacaattagtgcagccaggt

SEQ ID NO: 19 - DNA, 19 bp, synthetic construct
  source 1..19: mol_type = other DNA
agccacatcgctcagacac

SEQ ID NO: 20 - DNA, 19 bp, synthetic construct
  source 1..19: mol_type = other DNA
gcccaatacgaccaaatcc

SEQ ID NO: 21 - DNA, 20 bp, synthetic construct
  source 1..20: mol_type = other DNA
tgtgccatctgtctggagaa

SEQ ID NO: 22 - DNA, 20 bp, synthetic construct
  source 1..20: mol_type = other DNA
ttcctgtgaaaccggtgagt

SEQ ID NO: 23 - DNA, 20 bp, synthetic construct
  source 1..20: mol_type = other DNA
gtttgctggtgttgctgaaa

SEQ ID NO: 24 - DNA, 19 bp, synthetic construct
  source 1..19: mol_type = other DNA
tggcattgcacaggtacag

(19) United States
(12) Patent Application Publication
(10) Pub. No.: US 2026/0327281 A1
(43) Pub. Date: Oct. 22, 2026

(54) TARGETED PD-L1 DEGRADATION

(71) Applicant: Deutsches Krebsforschungszentrum Stiftung des öffentlichen Rechts, Heidelberg (DE)

(72) Inventors: Christof NIEHRS, Heidelberg (DE); Rui SUN, Heidelberg (DE)

(73) Assignee: Deutsches Krebsforschungszentrum Stiftung des öffentlichen Rechts, Heidelberg (DE)

(21) Appl. No.: 19/109,528

(22) PCT Filed: Sep. 8, 2023
(86) PCT No.: PCT/EP2023/074709

§ 371 Date: Mar. 6, 2025

(30) Foreign Application Priority Data
Sep. 9, 2022 (EP) 22194882.1

(51) Int. Cl. C07K 14/47; A61K 38/00; A61K 40/11; A61K 40/31; A61K 40/32; A61K 45/06; A61P 35/00; C07K 14/705; C12N 9/64; G01N 33/5751

(52) U.S. Cl. CPC C07K 14/4705; A61K 40/11; A61K 40/31; A61K 40/32; A61K 45/06; A61P 35/00; C07K 14/70521; C12N 9/6424; C12Y 304/21075; G01N 33/5751; A61K 38/00; C07K 2319/00; G01N 2333/70532

(57) ABSTRACT

The present invention relates to a binding polypeptide comprising a first binding domain binding a transmembrane E3 ligase; and a second binding domain binding a disease-related polypeptide, wherein said first binding domain comprises a furin domain 1 and/or a furin domain 2 of an R-spondin (RSPO), wherein said first binding domain lacks wnt signaling activity and/or lacks bone morphogenetic protein (BMP) signal inhibiting activity; and to polynucleotides, host cells, medical uses, and methods related thereto.

The present invention relates to a binding polypeptide comprising a first binding domain binding a transmembrane E3 ligase; and a second binding domain binding a disease-related polypeptide, wherein said first binding domain comprises a furin domain 1 and/or a furin domain 2 of an R-spondin (RSPO), wherein said first binding domain lacks wnt signaling activity and/or lacks bone morphogenetic protein (BMP) signal inhibiting activity; and to polynucleotides, host cells, medical uses, and methods related thereto.

Targeted protein degradation (TPD) is a rapidly progressing field that has broadened the scope of therapeutic targets to include historically undruggable proteins and overcome drug resistance (cf. e.g. Sun et al, Signal Transduct Target Ther 4:64 doi: 10.1038/s41392-019-0101-6 (2019)). Different from inhibiting the function of the protein of interest (POI) by traditional small-molecule drugs and antibody-based modalities, TPD induces the degradation of the targeted protein. Among various TPD platforms, proteolysis-targeting chimeras (PROTACs) have been successfully applied in the degradation of a variety of POIs implicated in multiple diseases, including cancer, infectious diseases, and neurodegenerative diseases (He et al., Front. Cell Dev. Biol 9:685106, doi: 10.3389/fcell.2021.685106 (2021)).

ZNRF3/RNF43 are single transmembrane E3 ligases with well-structured extracellular domains and functional intracellular RING domains (Zebisch et al., Nature comm 4:2787, doi: 10.1038/ncomms3787 (2013)). Their ligands R-spondins (RSPOs) are a family of four secreted stem cell growth factors with crucial implications in multiple biological processes, ranging from development to cancer (de Lau et al., Genome Biology 13:242 (2012); de Lau et al., Genes Dev 28:305, doi: 10.1101/gad.235473.113 (2014)). Recent studies revealed that among the four RSPOs, RSPO2 and RSPO3 are bi-functional ligands, activating WNT signaling and inhibiting BMP signaling (Lee et al., Nature comm 11:5570, doi: 10.1038/s41467-020-19373-w (2020); Sun et al., Cell rep 36:109559, doi: 10.1016/j.celrep.2021.109559 (2021)). They do so by employing ZNRF3/RNF43 to target distinct substrates.

ZNRF3 and RNF43 ubiquitinate WNT receptors Frizzled and LRP6, leading to their internalization and subsequential lysosomal degradation (Hao et al., Nature 485:195, doi: 10.1038/nature11019 (2012); Koo et al., Nature 488:665, doi: 10.1038/nature11308 (2012)). Besides WNT signaling activation, RSPO2 and RSPO3 also inhibit BMP signaling. Notably, the WNT agonistic function of R-spondins is associated with tumorigenesis (ter Stege et al. (2021), Oncogene 40 (47): 6469), limiting their therapeutic usability.

The programmed death-ligand 1 (PD-L1) protein, also known as CD274, is a 40 kDa transmembrane protein known to bind to the PD-1 receptor found on activated T cells and has been suggested to be involved in the control of the proliferation of CD8+ T cells. In accordance, PD-L1 antagonists have been proposed for blocking PD-L1 interaction with PD-1, thereby promoting tumor-directed T-cell activation in cancer treatment; e.g. Atezolizumab is a therapeutic monoclonal antibody targeting PD-L1.

In view of the above, there is still a need for improved treatments of cancer, in particular by activating and/or maintaining an immune response to cancer cells. The technical problem underlying the present invention may be seen as the provision of means and methods for complying with the aforementioned need. The technical problem is solved by the embodiments characterized in the claims and herein below.

In accordance, the present invention relates to a binding polypeptide comprising a first binding domain binding a transmembrane E3 ligase; and a second binding domain binding a disease-related polypeptide, preferably binding Programmed Death-Ligand 1 (PD-L1).

In general, terms used herein are to be given their ordinary and customary meaning to a person of ordinary skill in the art and, unless indicated otherwise, are not to be limited to a special or customized meaning. As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” preferably refer to “comprising one or more”, i.e. are equivalent to “comprising at least one”. In accordance, expressions relating to one item of a plurality, unless otherwise indicated, preferably relate to at least one such item, more preferably a plurality thereof; thus, e.g. identifying “a cell” relates to identifying at least one cell, preferably to identifying a multitude of cells.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

The methods specified herein below, preferably, are in vitro methods. The method steps may, in principle, be performed in any arbitrary sequence deemed suitable by the skilled person, but preferably are performed in the indicated sequence; also, one or more, preferably all, of said steps may be assisted or performed by automated equipment. Moreover, the methods may comprise steps in addition to those explicitly mentioned above.

As used herein, the term “standard conditions”, if not otherwise noted, relates to IUPAC standard ambient temperature and pressure (SATP) conditions, i.e. preferably, a temperature of 25° C. and an absolute pressure of 100 kPa; also preferably, standard conditions include a pH of 7. Moreover, if not otherwise indicated, the term “about” relates to the indicated value with the commonly accepted technical precision in the relevant field, preferably relates to the indicated value±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1% by weight, most preferably less than 0.1% by weight of non-specified component(s).

The term “binding” is understood by the skilled person; preferably, the term relates to an interaction of two molecules with a dissociation constant K_{D} of at most 10⁻⁶ mol/l, more preferably of at most 10⁻⁷ mol/l, even more preferably at most 10⁻⁸ mol/l, most preferably at most 10⁻⁹ mol/l. The term “specific binding” is also understood by the skilled person. Preferably, specific binding relates to a binding in which the affinity of the binding polypeptide to its cognate binding partner as specified elsewhere herein is at least tenfold, preferably at least 100 fold, more preferably at least 1000 fold, higher than for any non-cognate binding partner. Accordingly, the dissociation constant (K_{D}) of any binding polypeptide/non-cognate binding partner complex preferably is at least 10⁻⁶ mol/l, more preferably at least 10⁻⁵ mol/l, most preferably at least 10⁻⁴ mol/l.

The degree of identity (e.g. expressed as “% identity”) between two biological sequences, preferably DNA, RNA, or amino acid sequences, can be determined by algorithms well known in the art. Preferably, the degree of identity is determined by comparing two optimally aligned sequences over a comparison window, where the fragment of sequence in the comparison window may comprise additions or deletions (e.g., gaps or overhangs) as compared to the sequence it is compared to for optimal alignment. The percentage is calculated by determining, preferably over the whole length of the polynucleotide or polypeptide, the number of positions at which the identical residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman (1981), by the homology alignment algorithm of Needleman and Wunsch (1970), by the search for similarity method of Pearson and Lipman (1988), by computerized implementations of these algorithms (e.g. BLAST, GAP, BESTFIT, PASTA, or TFASTA), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. More preferably, the Basic Local Alignment Search Tool (BLAST) implementation is used with default parameter values for alignment. In the context of biological sequences referred to herein, the term “essentially identical” indicates a % identity value of at least 80%, preferably at least 90%, more preferably at least 98%, most preferably at least 99%. As will be understood, the term essentially identical includes 100% identity. The aforesaid applies to the term “essentially complementary” mutatis mutandis.

The term “fragment” of a biological macromolecule, preferably of a polynucleotide or polypeptide, is used herein in a wide sense relating to any sub-part, preferably subdomain, of the respective biological macromolecule comprising the indicated sequence, structure and/or function. Thus, the term includes sub-parts generated by actual fragmentation of a biological macromolecule, but also sub-parts derived from the respective biological macromolecule in an abstract manner, e.g. in silico. Thus, as used herein, an Fc or Fab fragment, but also e.g. a single-chain antibody, a bispecific antibody, and a nanobody may be referred to as fragments of an immunoglobulin.

Unless specifically indicated otherwise herein, the compounds specified, in particular the polynucleotides and polypeptides, may be comprised in larger structures, e.g. may be covalently or non-covalently linked to further sequences, carrier molecules, retardants, and other excipients. In particular, polypeptides as specified may be comprised in fusion polypeptides comprising further peptides, which may serve e.g. as a tag for purification and/or detection, as a linker, or to extend the in vivo half-life of a compound. The term “detectable tag” refers to a stretch of amino acids which are added to or introduced into the fusion polypeptide; preferably, the tag is added C- or N-terminally to the fusion polypeptide. Said stretch of amino acids preferably allows for detection of the polypeptide by an antibody which specifically recognizes the tag; or it preferably allows for forming a functional conformation, such as a chelator; or it preferably allows for visualization, e.g. in the case of fluorescent tags. Preferred detectable tags are the Myc-tag, FLAG-tag, 6-His-tag, HA-tag, GST-tag or a fluorescent protein tag, e.g. a GFP-tag. These tags are all well known in the art. Other further peptides preferably comprised in a fusion polypeptide comprise further amino acids or other modifications which may serve as mediators of secretion, as mediators of blood-brain-barrier passage, as cell-penetrating peptides, and/or as immune stimulants. Further polypeptides or peptides to which the polypeptides may be fused are signal and/or transport sequences, e.g. an IL-2 signal sequence, and linker sequences.

The term “polypeptide”, as used herein, refers to a molecule comprising several, typically at least 20, amino acids that are covalently linked to each other by peptide bonds. Molecules consisting of less than 20 amino acids covalently linked by peptide bonds are usually considered to be “peptides”. Preferably, the polypeptide comprises of from 50 to 1000, more preferably of from 100 to 1000, still more preferably of from 200 to 500, most preferably of from 250 to 400 amino acids. The polypeptide may be a complex of more than one amino acid chains, i.e. may be a multimer, e.g. a dimer, a trimer, and the like; in such case, the complex of more than one amino acid chains may also be referred to as an “polypeptide oligomer” or as a “protein complex”. Preferably, the complex of more than one amino acid chains is a hetero-multimer, more preferably a hetero-dimer, preferably comprising at least one first binding domain and at least one second binding domain in a non-covalent complex. Also, the polypeptide may comprise additional, non-peptidic structures, such as at least one glycosylation, lipid conjugation, and the like. More preferably, the polypeptide as specified, in particular the binding polypeptide, comprises all structural components as indicated comprised in one continuous covalent peptide chain, thus, the polypeptide, in particular the binding polypeptide, preferably is or is comprised in a fusion polypeptide. Unless specifically indicated otherwise, reference to specific polypeptides herein preferably includes polypeptide variants.

As used herein, the term “polypeptide variant” relates to any chemical molecule comprising at least one polypeptide as specified herein, having the indicated biological activity, but differing in structure from said specific polypeptide. Preferably, the polypeptide variant comprises a polypeptide having a contiguous amino acid sequence corresponding to at least 50%, preferably at least 75%, more preferably at least 85%, even more preferably at least 90%, most preferably at least 95%, of the amino acid sequence of the polypeptide specifically indicated, in particular of SEQ ID NO:7. Moreover, it is to be understood that a polypeptide variant as referred to in accordance with the present invention shall have an amino acid sequence which differs due to at least one amino acid substitution, deletion and/or addition, wherein the amino acid sequence of the variant is still, preferably, at least 70%, more preferably at least 80%, even more preferably at least 90%, even more preferably at least 95%, still more preferably at least 98%, most preferably at least 99%, identical with the amino acid sequence of the specific polypeptide, in particular SEQ ID NO:7 or 9. In view of the above, % identity values indicated herein below preferably are lower limits of % identity, and the required % identity may be higher, e.g. having the aforesaid values. The degree of identity between two amino acid sequences can be determined by algorithms well known in the art and as described herein above. Polypeptide variants referred to above may be allelic variants or any other species specific homologs, paralogs, or orthologs. Moreover, the polypeptide variants referred to herein include fragments of the specific polypeptides or the aforementioned types of polypeptide variants as long as these fragments and/or variants have the biological activity as specified. Such fragments may be or may be derived from, e.g., degradation products or splice variants of the polypeptides. Further included are variants which differ due to posttranslational modifications such as phosphorylation, glycosylation, ubiquitinylation, sumoylation, or myristylation, by including non-natural amino acids, and/or by being peptidomimetics. The above applies to domains of the polypeptide described herein mutatis mutandis and independently, i.e. the first binding domain and the second binding domain may be variant domains, still having the indicated activities, in particular binding activities, as specified herein below. Thus, the first binding domain may e.g. be a variant of a polypeptide comprising Furin 1 and Furin domain 2 of an R-spondin, e.g. may comprise an amino acid sequence at least 80% identical to SEQ ID NO:2, and the second binding domain may be a polypeptide variant of an extracellular IgV-like domain of a PD-1 polypeptide, e.g. may comprise an amino acid sequence at least 85% identical to SEQ ID NO:3.

The term “polynucleotide”, as used herein, refers to a linear or circular nucleic acid molecule. The polynucleotide of the present invention shall be provided, preferably, either as an isolated polynucleotide (i.e. isolated from its natural context) or in genetically modified form, preferably comprising at least one heterologous sequence. The term encompasses single- as well as double-stranded polynucleotides. Moreover, comprised are also chemically modified polynucleotides including naturally occurring modified polynucleotides such as glycosylated or methylated polynucleotides or artificially modified derivatives such as biotinylated polynucleotides, locked nucleic acids, and the like. The polynucleotides of the invention have the activity of encoding a binding polypeptide or at least one binding domain thereof as specified herein. Methods for testing whether a given polynucleotide has the aforesaid biological activity are known in the art and are described herein below. Unless specifically indicated otherwise, reference to specific polynucleotides herein preferably includes polynucleotide variants.

The term “polynucleotide variant”, as used herein, relates to a variant of a polynucleotide referred to herein comprising a nucleic acid sequence characterized in that the sequence can be derived from the aforementioned specific nucleic acid sequence by at least one nucleotide substitution, addition and/or deletion, wherein the polynucleotide variant shall have the biological activity as specified for the specific polynucleotide. Preferably, said polynucleotide variant is an ortholog, a paralog, or another homolog of the specific polynucleotide. Also preferably, said polynucleotide variant is or is derived from a non-naturally occurring allele of the specific polynucleotide. Polynucleotide variants also encompass polynucleotides comprising a nucleic acid sequence which is capable of hybridizing to the aforementioned specific polynucleotides, preferably, under stringent hybridization conditions. These stringent conditions are known to the skilled worker and can be found in Current Protocols in Molecular Biology, John Wiley & Sons, N. Y. (1989), 6.3.1-6.3.6. The skilled worker knows how to determine the hybridization conditions required by referring to textbooks such as the textbook mentioned above, or the textbooks indicated elsewhere herein. Alternatively, polynucleotide variants are obtainable by PCR-based techniques such as mixed oligonucleotide primer-based amplification of DNA. Further, variants include polynucleotides comprising nucleic acid sequences which are at least 70%, preferably at least 80%, more preferably at least 90%, even more preferably at least 95%, still more preferably at least 98%, most preferably at least 99%, identical to the specifically indicated nucleic acid sequences. Moreover, also encompassed are polynucleotides which comprise nucleic acid sequences encoding amino acid sequences which are at least 70%, more preferably at least 80%, even more preferably at least 90%, even more preferably at least 95%, still more preferably at least 98, most preferably at least 99%, identical to the amino acid sequences specifically indicated. The percent identity values are, preferably, calculated over the entire amino acid or nucleic acid sequence region, preferably as specified herein above. The polynucleotides of the present invention either consist of, essentially consist of, or comprise the aforementioned nucleic acid sequences. Thus, they may contain further nucleic acid sequences as well. Specifically, the polynucleotides of the present invention may encode fusion proteins wherein one partner of the fusion protein is a polypeptide being encoded by a nucleic acid sequence recited above. Also, the polynucleotide may be comprised in an expression construct and/or a vector.

The term “expression construct”, as used herein, refers to a heterologous polynucleotide comprising the aforementioned polynucleotide as well as nucleic acid sequences required for expression of the polynucleotide. Typically, such additional nucleic acid sequences, which preferably are heterologous to the polynucleotide encoding the binding polypeptide or at least one binding domain thereof, may be promoter sequences, regulatory sequences and/or transcription termination sequences, such as terminators. Expression of the polynucleotide comprises transcription of the polynucleotide into an RNA. Regulatory elements ensuring expression in cells, in particular eukaryotic cells, preferably mammalian cells, are well known in the art. They, preferably, comprise regulatory sequences ensuring initiation of transcription and, optionally, poly-A signals ensuring termination of transcription and stabilization of the transcript. Additional regulatory elements may include transcriptional as well as translational enhancers. Possible regulatory elements permitting expression in prokaryotic host cells comprise, e.g., the lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer, or a globin intron in mammalian and other animal cells. Moreover, inducible expression control sequences may be used in an expression construct encompassed by the present invention. Such inducible constructs may comprise tet or lac operator sequences or sequences inducible by heat shock or other environmental factors. Suitable expression control sequences are well known in the art. Beside elements which are responsible for the initiation of transcription such regulatory elements may also comprise transcription termination signals, such as the SV40-poly-A site or the tk-poly-A site, downstream of the polynucleotide. Preferably, the expression construct is a eukaryotic expression construct, i.e. an expression construct comprising all elements required for expression, preferably inducible expression, in a eukaryotic host cell. The expression construct may, however, also be a bacterial expression construct for producing the binding polypeptide in bacterial cells.

The term “vector”, preferably, encompasses phage, plasmid, viral or retroviral vectors as well as artificial chromosomes, such as bacterial or yeast artificial chromosomes. Moreover, the term also relates to targeting constructs which allow for random or site-directed integration of the targeting construct into genomic DNA. Such target constructs, preferably, comprise DNA of sufficient length for either homologous or heterologous recombination as described in detail below. The vector encompassing the polynucleotide of the present invention, preferably, further comprises selectable markers for propagation and/or selection in a host. The vector may be incorporated into a host cell by various techniques well known in the art. For example, a plasmid vector can be introduced in a precipitate such as a calcium phosphate precipitate or rubidium chloride precipitate, or in a complex with a charged lipid or in carbon-based clusters, such as fullerenes. Alternatively, a plasmid vector may be introduced by heat shock or electroporation techniques. Should the vector be a virus, it may be packaged in vitro using an appropriate packaging cell line prior to application to host cells. Viral vectors, in particular retroviral vectors, may be replication competent or replication defective. In the latter case, viral propagation generally will occur only in complementing host/cells. More preferably, in the vector of the invention the polynucleotide is operatively linked to expression control sequences allowing expression in prokaryotic or eukaryotic cells or isolated fractions thereof, i.e. preferably, the polynucleotide is comprised in an expression vector. In this context, suitable expression vectors are known in the art such as Okayama-Berg cDNA expression vector pcDV1 (Pharmacia), pBluescript (Stratagene), pCDM8, pRc/CMV, pcDNA1, pcDNA3 (In Vitrogene) or pSPORT1 (GIBCO BRL). Preferably, the vector is an expression vector, a gene transfer and/or targeting vector. Expression vectors derived from viruses such as retroviruses, vaccinia virus, or adeno-associated virus may be used for delivery of polynucleotides or vectors into targeted cell population. Methods which are well known to those skilled in the art can be used to construct recombinant viral vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994).

In view of the above, the skilled person understands that references herein to a gene or its gene product include polynucleotide variants of the gene and its transcription product(s), i.e. in particular alleles, homologs, and mutants of the gene, as well as e.g. transcript variants, such as splice variants and RNA editing variants of the RNA gene products. More preferably, reference to a gene relates to a gene and its naturally occurring alleles. Also, reference to a polypeptide gene product includes polypeptide variants as specified herein above, in particular isoforms and/or muteins having the indicated degree of sequence identity and having the indicated biological activity. For those genes or gene products referred to by a Genbank Acc No or a similar designation, the respective sequence is herewith included to this description by reference. Unless indicated otherwise, database entries relate to the state of the respective database entry on the day before the filing date of the present application.

The term “R-spondin” is known to the skilled person to relate to a family of roof plate-specific spondins, reviewed e.g. by de Lau et al., Genome Biology 13:242 (2012). Thus, the R-spondin may be one of R-spondins 1 to 4, preferably one of human R-spondins 1 to 4. Preferably, the R-spondin is R-spondin 2 or R-spondin 3. The term “R-spondin 2” is known to the skilled person. The human R-spondin 2 polypeptide has several isoforms, the amino acid sequence e.g. of isoform 1 precursor being provided as Genbank Acc No. NP_848660.3, SEQ ID NO: 12. In accordance, the term “R-spondin 2”, as used herein, preferably relates to the aforesaid human R-spondin 2, or to a polypeptide having an amino acid sequence at least 70%, preferably at least 80%, more preferably at least 90%, even more preferably at least 95%, still more preferably at least 98%, most preferably at least 99% identical to the amino acid sequence of said human R-spondin 2. Preferably, the R-spondin 2 is human R-spondin 2 or a homolog thereof, preferably a vertebrate homolog, more preferably a mammalian homolog. Homologs of R-spondin 2 are known e.g. from HomoloGene database entry 18235; moreover R-spondin 2 homologues in other species can be identified by sequence comparison, in particular by determining the degree of identity as described elsewhere herein. Thus, the R-spondin 2 preferably is R-spondin 2 of a human, a chimpanzee, a rhesus monkey, a rat, a mouse, a cattle, a dog, a chicken, a zebrafish, or from a western clawed frog, more preferably of a human. The term “R-spondin 3” is also known to the skilled person. The amino acid sequence of human R-spondin 3 precursor is available e.g. as Genbank Acc. No. NP_116173.2, SEQ ID NO:2. In accordance, the term “R-spondin 3”, as used herein, preferably relates to the aforesaid human R-spondin 3, or to a polypeptide having an amino acid sequence at least 50%, preferably at least 60%, more preferably at least 70%, even more preferably at least 80%, still more preferably at least 90%, most preferably at least 95% identical to the amino acid sequence of said human R-spondin 3. Preferably, the R-spondin 3 is human R-spondin 3 or a homolog thereof, preferably a vertebrate homolog, more preferably a mammalian homolog. Homologs of R-spondin 3 are known e.g. from HomoloGene database entry 12484; moreover R-spondin 3 homologues in other species can be identified by sequence comparison, in particular by determining the degree of identity as described elsewhere herein. Thus, the R-spondin 3 preferably is R-spondin 3 of a human, a chimpanzee, a rhesus monkey, a rat, a mouse, a cattle, a chicken, a zebrafish, or from a western clawed frog, more preferably of a human.

The term “binding domain”, as used herein, relates to a sub-structure of a binding polypeptide having the indicated biological binding activity; preferably, said binding is specific binding. Preferably, the binding domain comprises, more preferably consists of, a continuous amino acid chain having the indicated binding activity. Preferably, the binding domain only has the indicated binding activity, i.e. preferably is devoid of biological activities other than those specifically indicated, although such additional biological activities may have been present in the protein the binding domain is derived from. Thus, preferably the binding domain, in particular the first binding domain, more preferably both binding domains, most preferably the binding polypeptide, has/have no wnt-signaling activity and/or no bone morphogenetic protein (BMP) signal inhibiting activity, more preferably has/have no signaling activity. As specified herein, the binding polypeptide comprises at least a first binding domain and a second binding domain, wherein the first binding domain has the biological activity of binding a transmembrane E3 ligase; and a second binding domain having the biological activity of binding a disease-related polypeptide.

The first binding domain has the biological activity of binding a transmembrane E3 ligase. The term “transmembrane E3 ligase” is understood by the skilled person to relate to the transmembrane members of the family of E3 ligases (EC 2.3.2.27). Preferably, the transmembrane E3 ligase is a RING-finger E3 ligase. Preferably, the transmembrane E3 ligase is expressed by a target cell. Preferably, the transmembrane E3 ligase is a ZNRF3 E3 ligase, preferably human ZNRF3 E3 ligase, more preferably having the amino acid sequence of Genbank Acc No. NP_001193927.1, SEQ ID NO:10, or one of its isoforms; also preferably, the transmembrane E3 ligase is an RNF43 E3 ligase, preferably human RNF43 E3 ligase, more preferably having the amino acid sequence of Genbank Acc No. NP_060233.3, SEQ ID NO:11, or one of its isoforms. Polypeptides having the activity of binding transmembrane E3 ligases are known in the art and include in particular R-spondins as specified herein above. Thus, preferably, the first binding domain comprises a sub-portion of an R-spondin, preferably R-spondin 2, having the aforesaid activity. Thus, the first binding domain preferably comprises a furin domain 1 and/or a furin domain 2 of an R-spondin (RSPO), wherein said furin domain 2 is devoid of wnt-activating activity. Preferably furin domain 1 corresponds to amino acids 37 to 84 of SEQ ID NO:12 or corresponding amino acids of an other-spondin polypeptide, and/or furin domain 2 corresponds to amino acids 90 to 134 of SEQ ID NO:12 or corresponding amino acids of an other r-spondin polypeptide. More preferably, the furin domain 2 comprises an exchange of amino acid F109 for a non-identical amino acid, preferably comprises an F109A amino acid exchange, wherein amino acid position 109 is the position with this number in human R-spondin 2 as specified herein above, or a corresponding amino acid position in one of the other R-spondins. Other amino acid exchanges abolishing wnt signaling in R-spondins and/or furin domains are known in the art and the skilled person knows how to put such amino acid exchanges into practice in the context of a first binding domain; thus, a further preferred amino acid exchanges having the aforesaid effect is an exchange of amino acid F105 for a non-identical amino acid, preferably an F105A amino acid exchange, wherein amino acid position 105 is the position with this number in human R-spondin 2 as specified herein above, or a corresponding amino acid position in one of the other R-spondins. Preferably, the first binding domain comprises an amino acid sequence at least 80% identical to SEQ ID NO:1. More preferably, the first binding domain comprises furin domain 1 and furin domain 2 of RSPO2. Thus, the first binding domain more preferably comprises an amino acid sequence at least 80% identical to SEQ ID NO:2. More preferably, the first binding domain consists of an amino acid sequence at least 80% identical to SEQ ID NO:1, more preferably at least 80% identical to SEQ ID NO: 2. Thus, the first binding domain preferably lacks a TSP1 domain; also preferably, the first binding domain consists of said furin domain 1 and/or said furin domain 2, wherein said furin domain 2 preferably comprises the aforesaid F109A amino acid exchange.

The term “subject”, as referred to herein, relates to a vertebrate animal, preferably a mammal, in particular a livestock, companion, or laboratory animal. More preferably, the subject is a human. Preferably, the subject has been diagnosed to suffer from a disease as specified herein below and/or was diagnosed to be at risk of developing a disease. More preferably, the subject has been diagnosed to suffer from cancer and/or was diagnosed to be at risk of developing a relapse and/or metastases.

The term “disease” is used herein in a broad sense and preferably relates to any pathological state of a subject, preferably requiring treatment and more preferably being known or suspected to be treatable by a binding polypeptide as specified herein. In accordance, the disease preferably is a pathological state caused or aggravated by (over) expression of at least one disease-related polypeptide as specified elsewhere herein. Thus, the disease may in particular be caused or aggravated by overexpression of a polypeptide at non-physiological levels and/or by expression of a mutein of a polypeptide of the subject, which mutein may e.g. have an increased activity, a neoactivty, and/or may inhibit physiological regulation. Preferably, the disease is caused or aggravated by immune system malfunction, e.g. may be cancer or autoimmune disease. Preferably, the disease is caused or aggravated by overexpression of at least one immune checkpoint inhibitor, preferably of PD-L1. More preferably, the disease is cancer, still more preferably PD-L1 expressing cancer.

The term “cancer”, as used herein, relates to a disease of an animal, including man, characterized by uncontrolled growth by a group of body cells (“cancer cells”). This uncontrolled growth may be accompanied by intrusion into and destruction of surrounding tissue (infiltration) and possibly spread of cancer cells to other locations in the body (metastasis). Preferably, also included by the term cancer is a recurrence of a cancer (relapse). Thus, preferably, the cancer is a solid cancer, a metastasis, or a relapse thereof. Also preferably, the cancer is a non-solid cancer, in particular a leukemia, in particular a relapse or an advanced stage leukemia. Preferably, the cancer is selected from the list consisting of acute myeloid leukemia (AML), acute lymphoblastic leukemia, adrenocortical carcinoma, aids-related lymphoma, anal cancer, appendix cancer, astrocytoma, atypical teratoid, basal cell carcinoma, bile duct cancer, bladder cancer, brain stem glioma, breast cancer, burkitt lymphoma, carcinoid tumor, cerebellar astrocytoma, cervical cancer, chordoma, chronic lymphocytic leukemia, chronic myelogenous leukemia, colon cancer, colorectal cancer, craniopharyngioma, endometrial cancer, ependymoblastoma, ependymoma, esophageal cancer, extracranial germ cell tumor, extragonadal germ cell tumor, extrahepatic bile duct cancer, fibrosarcoma, gallbladder cancer, gastric cancer, gastrointestinal stromal tumor, gestational trophoblastic tumor, hairy cell leukemia, head and neck cancer, hepatocellular cancer, hodgkin lymphoma, hypopharyngeal cancer, hypothalamic and visual pathway glioma, intraocular melanoma, kaposi sarcoma, laryngeal cancer, medulloblastoma, medulloepithelioma, melanoma, merkel cell carcinoma, mesothelioma, mouth cancer, multiple endocrine neoplasia syndrome, multiple myeloma, mycosis fungoides, nasal cavity and paranasal sinus cancer, nasopharyngeal cancer, neuroblastoma, non-hodgkin lymphoma, non-small cell lung cancer, oral cancer, oropharyngeal cancer, osteosarcoma, ovarian cancer, ovarian epithelial cancer, ovarian germ cell tumor, ovarian low malignant potential tumor, pancreatic cancer, papillomatosis, paranasal sinus and nasal cavity cancer, parathyroid cancer, penile cancer, pharyngeal cancer, pheochromocytoma, pituitary tumor, pleuropulmonary blastoma, primary central nervous system lymphoma, prostate cancer, rectal cancer, renal cell cancer, retinoblastoma, rhabdomyosarcoma, salivary gland cancer, sézary syndrome, small cell lung cancer, small intestine cancer, soft tissue sarcoma, squamous cell carcinoma, squamous neck cancer, testicular cancer, throat cancer, thymic carcinoma, thymoma, thyroid cancer, urethral cancer, uterine sarcoma, vaginal cancer, vulvar cancer, waldenström macroglobulinemia, and wilms tumor. More preferably, the cancer is a melanoma.

The second binding domain has the biological activity of binding to a disease-related polypeptide, preferably an extracellular domain thereof, preferably has the activity of binding to Programmed Death-Ligand 1 (PD-L1), preferably its extracellular domain.

The term “disease-related polypeptide” is understood by the skilled person to include any and all polypeptides causally involved in the etiology of disease; thus, the disease-related polypeptide preferably is known to be overexpressed in disease and/or is known to be a mutein of a polypeptide normally present in a subject, wherein preferably said overexpression or expression of a mutein is causally linked to disease. Thus, reduction of the amount of the disease-related polypeptide preferably causes, more preferably is known to cause, at least amelioration of disease. The disease-related polypeptide preferably is membrane-bound, preferably to the cytoplasmic membrane of a target cell. Thus, the disease-related polypeptide preferably comprises at least one transmembrane domain, i.e., preferably is a transmembrane polypeptide. Also preferably, the disease-related polypeptide comprises at least one extracellular domain. The disease-related polypeptide may, however, also be a non-transmembrane polypeptide; preferably, in such case the disease-related polypeptide is a membrane-bound polypeptide, in particular a lipid anchored polypeptide, or is an extracellular, preferably soluble, polypeptide which is non-covalently bound to a membrane, in particular a cytoplasmic membrane. Non-covalent binding of a soluble polypeptide to a membrane may be mediated by protein-protein interaction, protein-lipid interaction, protein-glycan interaction, metal ions, or the like. Preferably, the disease-related polypeptide is a regulatory polypeptide contributing to regulation (i) of cellular proliferation, (ii) of an immune response, in particular a cellular immune response, and/or (iii) of intercellular communication. Thus, the disease-related polypeptide may in particular be a T cell regulator polypeptide or an immune checkpoint polypeptide. In particular, the disease-related polypeptide may be PD-L1.

Preferably, the disease-related polypeptide is Programmed Death-Ligand 1 (PD-L1). The terms “Programmed Death-Ligand 1” and “PD-L1” relate to the group of polypeptides known under this designation, which may also be referred to as “CD274” or “B7-H1”. Preferably, PD-L1 is expressed by a target cell. Preferably, PD-L1 is human PD-L1, more preferably having the amino acid sequence of Genbank Acc No. NP_054862.1, SEQ ID NO: 13, or one of its isoforms.

Polypeptides and domains thereof having the activity of binding to a disease-related polypeptide are known in the art and are selected by the skilled person in particular based on the disease-related polypeptide intended to be targeted. Thus, the second binding domain may in particular be a binding domain of a, preferably naturally occurring, interaction partner of the disease-related polypeptide. Thus, in case the disease-related polypeptide is a receptor, the second binding domain may e.g. comprise a binding domain of said receptor's cognate ligand or a derivative thereof having the aforesaid binding activity. Also, the second binding domain may comprise an aptamer, a spiegelmer, a designed ankyrin repeat (darpin) domain, a Kunitz type domain, an antibody, in particular a single chain or single domain antibody (cf. Hey et al. (2005), Trends Biotechnol. 23:514), or a derivative thereof having the aforesaid binding activity.

Polypeptides having the activity of binding to PD-L1 are known in the art and include in particular programmed cell death protein 1 (PD-1). The terms “programmed cell death protein 1” and “PD-1” relate to a member of the family of T cell regulators known to the skilled person under this designation, which may also be referred to as “CD279”. Preferably, PD-1 is human PD-1, more preferably comprising an amino acid sequence as shown in Genbank Acc No. NP_005009.2, SEQ ID NO: 14, or one of its isoforms. PD-1 comprises an extracellular IgV-like domain binding to the extracellular domain of PD-L1; thus, the second binding domain preferably comprises an extracellular IgV-like domain of PD-1 polypeptide, more preferably, a high-affinity variant thereof. In accordance, the second binding domain comprises an amino acid sequence at least 80% identical to SEQ ID NO:3 or 4. Also preferably, second binding domain comprises an amino acid sequence at least 75% identical to SEQ ID NO:5. Thus, in case the second binding domain comprises an amino acid sequence at least 80% identical to SEQ ID NO:3 or 4, it preferably also comprises an amino acid sequence at least 75% identical to SEQ ID NO:5. More preferably, the second binding domain consists of one of the aforesaid amino acid sequences.

The term “binding polypeptide”, as used herein, relates to a polypeptide as specified herein above having the structural elements and biological activity or activities as specified. Thus, the binding polypeptide comprises at least a first binding domain having the biological activity of binding a transmembrane E3 ligase; and a second binding domain having the biological activity of binding a disease-related polypeptide. Thus, the binding polypeptide has the biological activities of binding a transmembrane E3 ligase and binding a disease-related polypeptide. Preferably, said binding activities cause a transmembrane E3 ligase and a disease-related polypeptide to become interlinked at the cytoplasmic membrane of a target cell via the binding polypeptide, and preferably cause the disease-related polypeptide to be degraded by the cell.

In view of the description herein above, the binding polypeptide preferably only has the indicated binding activities, i.e. preferably is devoid of biological activities other than those specifically indicated, although such additional biological activities may have been present in the proteins the binding polypeptide is derived from. Thus, the binding polypeptide preferably has no wnt-signaling activity and/or no bone morphogenetic protein (BMP) signal inhibiting activity, more preferably has no signal modulating activity, preferably has no signaling activity. Also preferably, the binding polypeptide is devoid of additional sequences of the protein(s) it is derived from. Thus, the binding polypeptide preferably does not comprise a transmembrane domain and/or intracellular domain of a PD-1; also, the binding polypeptide preferably does not comprise an active TSP-1 domain of an R-spondin, i.e. in case the binding polypeptide comprises a TSP-1 domain, it preferably is a signaling inactive polypeptide variant. More preferably, the binding polypeptide preferably does not comprise an active TSP-1 domain of an R-spondin, more preferably does not comprise any at least 7 amino acids long amino acid sequence downstream of amino acid 143 of SEQ ID NO:12.

Nonetheless, the binding polypeptide may comprise amino acid sequences in addition to those specifically indicated, e.g. tag or flag sequences, a signal peptide, e.g. having the amino acid sequence of SEQ ID NO:8, or at least one linker sequence. Further amino acid sequences or other structural elements may be included to provide further binding activities, improved serum stability, tissue targeting, or the like. Preferably, however, the binding polypeptide does not comprise a transmembrane domain and/or an intracellular domain. Thus, the binding polypeptide preferably is a non-membrane integral polypeptide, more preferably is a soluble polypeptide.

Preferably, the binding polypeptide is a complex of more than one amino acid chains, i.e. preferably is a multimer, e.g. a dimer, a trimer, or the like; in such case, the complex of more than one amino acid chains may also be referred to as an “binding polypeptide oligomer” or as a “binding polypeptide complex”. Preferably, the complex of more than one amino acid chains is a hetero-multimer, more preferably a hetero-dimer, preferably comprising at least one first binding domain and at least one second binding domain in a non-covalent complex. Methods of providing appropriate complexes are known in the art, using e.g. appropriate ligand-receptor pairs, antibody-antigen interactions, and the like. On an exemplary basis, the interaction of biotin with streptavidin or an interaction pair derived therefrom may be used. Preferably, a colicin/immunity affinity pair is used for complex formation. As the skilled person understands, in case the binding polypeptide is a binding polypeptide complex, said complex may be formed in vitro, e.g. by mixing a first binding domain covalently coupled to a first partner of an affinity pair with a second binding domain coupled to a second partner of an affinity pair; the complex may, however, also be formed in vivo, e.g. by administering a first binding domain covalently coupled to a first partner of an affinity pair and further administering a second binding domain coupled to a second partner of an affinity pair. As will also be understood by the skilled person, it is also possible to use more than one second binding domains together with one first binding domain, e.g. by using a first binding domain covalently coupled to a first partner of an affinity pair in conjunction with a multitude of second binding domains, each of which is coupled to a second partner of an affinity pair; this way, more than one disease-related polypeptides can be targeted.

More preferably, in the binding polypeptide the first binding domain and the second binding domain are covalently connected, directly or indirectly. More preferably, the first binding domain and the second binding domain are comprised in a common polypeptide, i.e. preferably the first binding domain and the second binding domain together form a fusion polypeptide. Preferably, the first binding domain and the second binding domain are connected via a linker, i.e., preferably, the linker intervenes between said first binding domain and said second binding domain. The terms “linker”, “linker sequence”, and “linker peptide” are in principle known to the skilled person. The person skilled in the art knows how to select suitable linker peptides. Preferably, the linker comprises 1 to 20, more preferably 5 to 10, amino acids, which are preferably independently selected from the group consisting of Glycine (G), Proline (P), and Serine(S). A particularly preferred linker peptide comprises the amino acid sequence shown as SEQ ID NO:6. Preferably, the first binding domain lies N-terminal of the second binding domain in the binding polypeptide; thus, in case a linker is present, the order of structural elements in the binding polypeptide preferably is N-terminus, first binding domain, linker, second binding domain, C-terminus. As the skilled person understands, in case additional structural elements are present in the binding polypeptide, they are preferably attached N-terminal and/or C-terminal of the aforesaid preferred structure. Also, additional structural elements may be added with or without an intervening linker, wherein said optional linker may or may not be the same as the linker intervening the first and second binding domains.

In accordance with the above, the binding polypeptide preferably comprises an amino acid sequence at least 80% identical to SEQ ID NO:7 or 9. More preferably the binding polypeptide essentially consists of an amino acid sequence at least 80% identical to SEQ ID NO:7 or 9, most preferably the binding polypeptide consists of an amino acid sequence at least 80% identical to SEQ ID NO:7 or 9.

Preferably, the binding polypeptide is comprised in a composition of matter comprising further components; thus, the binding polypeptide may be comprised in a solution, in particular aqueous solution, which may additionally comprise e.g. at least one buffer, at least one salt, and/or any other component(s) deemed appropriate by the skilled person. Preferably, the composition is a pharmaceutical composition, said pharmaceutical composition preferably further comprising a pharmaceutically acceptable carrier. The terms “medicament” and “pharmaceutical composition” are used essentially interchangeably herein and are, in principle, known to the skilled person. As referred to herein, the terms preferably relate to any composition of matter comprising the specified active agent(s) as pharmaceutically active compound(s) and, optionally, one or more excipient. The pharmaceutically active compound(s) can be present in liquid or dry, e.g. lyophilized, form. It will be appreciated that the form and character of the pharmaceutical acceptable excipient, e.g. carrier or diluent, is dictated by the amount of active ingredient with which it is to be combined, the route of administration, and other well-known variables. The excipient(s) must be acceptable in the sense of being compatible with the other ingredients of the formulation and being not deleterious to the recipient thereof. The excipient employed may include a solid, a gel, or a liquid. Exemplary of solid carriers are lactose, terra alba, sucrose, talc, gelatin, agar, pectin, acacia, magnesium stearate, stearic acid, and the like. Exemplary of liquid carriers are phosphate buffered saline solution, physiological saline, Ringer's solutions, dextrose solution, Hank's solution, syrup, oil, water, emulsions, various types of wetting agents, and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pennsylvania. The excipient(s) is/are selected so as not to affect the biological activity of the combination. The excipient may, however, also be selected to improve uptake of the active agent into a cell, in particular a cancer cell.

The medicament may be administered by any route deemed appropriate, e.g. by a medical practitioner, preferably at a therapeutically effective dose. A therapeutically effective dose refers to an amount of the active compound which prevents, ameliorates or cures the symptoms accompanying a disease or condition referred to in this specification. Therapeutic efficacy and toxicity of a drug can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. The dosage regimen will be determined by the attending physician and by clinical factors. As is well known in the medical arts, dosages for any one patient may depend upon many factors, including type and severity of disease, the patient's size, age, the particular formulation of the medicament to be administered, sex, time and route of administration, general health, and other drugs being administered concurrently. The medicament referred to herein is, preferably, administered at least once, e.g. as a bolus. However, the medicament may be administered more than one time and, preferably, at least twice, e.g. permanently or periodically after defined time windows. Progress can be monitored by periodic assessment. Dosage recommendations may be indicated in the prescriber or user instructions in order to anticipate dose adjustments depending on the considered recipient. The medicament according to the present invention may comprise further active agents in addition to the aforementioned active agent(s). Preferably, the pharmaceutically active compounds according to the invention are to be applied together with at least one further drug and, thus, may be formulated together with this at least one further drug as a medicament. More preferably, in case of cancer treatment, said at least one further active agent is a chemotherapeutic agent or an immunotherapeutic agent, such as a T cell or an immune checkpoint modulator. Also, it is to be understood that the formulation of a pharmaceutical composition preferably takes place under GMP standardized conditions or the like in order to ensure quality, pharmaceutical safety, and effectiveness of the medicament.

Advantageously, it was found in the work underlying the present invention that the binding polypeptide described herein in the Examples induces degradation of PD-L1 as a model target polypeptide on target cells, thereby not only relieving PD-1 mediated immune checkpoint blockade, but also removing one of the factors mediating it, thereby improving treatment efficacy. The binding polypeptide was found to target the highly relevant immune checkpoint protein PD-L1 and caused its degradation. Importantly, PD-L1 degradation occurred in the pico-to low nanomolar range, indicating that the binding polypeptide is highly potent. Notably, ZNRF3 and RNF43, the exemplary transmembrane E3 ligases used in the Examples, show broad to ubiquitous expression across many tissues and cell lines (cf. e.g. www.ncbi.nlm.nih.gov/gene/84133 and -54894). Thus, the instant description provides an R-spondin binding domain, that (i) is WNT- and BMP signaling-disabled by introducing suitable mutations; (ii) is coupled to a suitable second binding polypeptide, and (iii) retains biological activity to bind to E3 ligases with high affinity and to be internalized and degraded. In accordance, the instant work provides a generally applicable, signaling-disabled R-spondin derived first binding domain for proximity-based protein degradation therapy.

The definitions made above apply mutatis mutandis to the following. Additional definitions and explanations made further below also apply for all embodiments described in this specification mutatis mutandis.

The present invention further relates to a polynucleotide encoding a binding polypeptide according to the present invention.

The polynucleotides of the present invention have been described herein above; as indicated, the polynucleotide may be comprised in an expression construct and/or a vector, i.e. preferably in an expression vector, all as described herein above as well.

The present invention also relates to a host cell comprising a binding polypeptide according to the present invention and/or a polynucleotide according to the present invention.

The term “cell”, as used herein, is understood by the skilled person as relating to the smallest structural unit in biology with the principal ability of self-replication. Thus, the term includes archeal, prokaryotic, and eukaryotic cells. The term “host cell” relates to a cell capable of transiently, preferably stably, maintaining a polynucleotide or vector as described herein, and/or expressing a binding polypeptide from an expression construct or expression vector as described herein; thus the host cell may be, e.g. a bacterial cell, in particular an Escherichia coli cell; or may be a eukaryotic cell, e.g. a cell of a subject to be treated with the binding polypeptide. The term “target cell” relates to a cell known or suspected to express at least one transmembrane E3 ligase and a disease-related polypeptide; preferably, the target cell is a cancer cell. Preferably, the target cell is a cell of a subject or a cultured cell derived therefrom.

The present invention also relates to a device comprising a binding polypeptide according to the present invention and/or a polynucleotide according to the present invention.

The term “device”, as used herein relates to a system of means comprising at least the means described, preferably operatively linked to each other and/or further means such as to allow administration of the compound or of the composition of the present invention. Preferred means for administering a binding polypeptide are well known in the art and are described herein above. How to link the means in an operating manner will depend on the type of means included into the device and on the kind of administration envisaged. Preferably, the means are comprised by a single device in such a case. Said device may accordingly include a delivery unit for the administration of the binding polypeptide and, optionally, a storage unit for storing said binding polypeptide until administration. However, it is also contemplated that the means of the current invention may appear as separate devices in such an embodiment and are, preferably, packaged together as a kit. The person skilled in the art will realize how to link the means without further ado. Preferred devices are those which can be applied without the particular knowledge of a specialized technician. In a preferred embodiment, the device is a syringe, more preferably with a needle, comprising the binding polypeptide. More preferably, the device is an intravenous infusion (IV) equipment comprising the binding polypeptide. Also preferably, the device is a tube or an endoscopic device comprising the preparation for flushing a site of administration, e.g. a heart, or further comprising a needle for topical application of the compound or composition, e.g. to tumor.

The resent invention also relates to a kit comprising a binding polypeptide according to the present invention and/or a polynucleotide according to the present invention, and optionally a means of administration.

The term “kit”, as used herein, refers to a collection of the aforementioned compounds, means or reagents which may or may not be packaged together. The components of the kit may be comprised by separate vials (i.e. as a kit of separate parts) or provided in a single vial, e.g. as a composition as specified herein above. In view of the above, the kit may e.g. comprise (i) a first binding domain coupled to a first partner of an affinity pair and (ii) at least one second binding domain coupled to a second partner of an affinity pair, e.g. in separate vials, or as a pre-formed complex. The housing of the kit in an embodiment allows translocation of the compounds of the kit, in particular common translocation; thus, the housing may in particular be a transportable container comprising all specified components. Moreover, it is to be understood that the kit of the present invention may be used for practicing the methods referred to herein above. It is preferably envisaged that all components are provided in a ready-to-use manner for practicing the methods referred to above. Further, the kit preferably contains instructions for carrying out said methods. The instructions can be provided by a user's manual on paper or in electronic form. For example, the manual may comprise instructions for interpreting the results obtained when carrying out the aforementioned methods using the kit. Preferably, the kit is adapted for use in a method of the present invention, more preferably is adapted to comprise all reagents required to perform said method or methods.

The present invention also relates to a method for killing cancer cells, comprising contacting said cancer cells with a binding polypeptide according to the present invention and/or a polynucleotide according to the present invention.

The method for killing cancer cells of the present invention, preferably, is an in vitro method. The method may, however, also be performed in vivo, e.g. as part of a method of treating and/or preventing cancer as specified herein below. The method may comprise steps in addition to those explicitly mentioned above. For example, further steps may relate, e.g., to identifying cancer cells, for determining expression of at least one transmembrane E3 ligase and/or a disease-related polypeptide on said cell, and the like. Moreover, one or more of said steps may be performed or assisted by automated equipment. Thus, the method may in particular be practiced on a cell, preferably a target cell, expressing a disease-related polypeptide. Also preferably, the method is practiced on a cell, preferably a target cell, overexpressing at least one transmembrane E3 ligase, in particular ZNRF3 and/or RNF43; thus, preferably, the method further comprises contacting said cancer cells with an agent providing a transmembrane E3 ligase.

The term “agent providing a transmembrane E3 ligase” includes each and every agent, which, when contacted to a cell, preferably a target cell, causes at least one transmembrane E3 ligase to be present in said cell in an amount increased compared to a cell not contacted to said agent. Thus, the agent providing a transmembrane E3 ligase may be a transmembrane E3 ligase polypeptide, preferably comprised in liposomes which may fuse with the cytoplasmic membrane of a cell. More preferably, the agent providing a transmembrane E3 ligase is a polynucleotide encoding a transmembrane E3 ligase, more preferably an expression construct for a transmembrane E3 ligase.

The present invention also relates to a binding polypeptide according to the present invention and/or a polynucleotide according to the present invention, for use in medicine; and to a use of the binding polypeptide according to the present invention and/or the polynucleotide according to the present invention, for the manufacture of a medicament.

Further, the present invention relates to a binding polypeptide according to the present invention and/or a polynucleotide according to the present invention, for use in treating and/or preventing cancer; and to a use of a binding polypeptide according to the present invention and/or a polynucleotide according to the present invention for the manufacture of a medicament for treating and/or preventing cancer.

The terms “treating” and “treatment” refer to an amelioration of a disease or disorder referred to herein or the symptoms accompanied therewith to a significant extent; as used herein, the terms include prevention of deterioration of a disease, disorder, or symptoms associated therewith. Said treating as used herein may also include an entire restoration of health with respect to the diseases or disorders referred to herein. It is to be understood that treating, as the term is used herein, may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001. Preferably, the treatment shall be effective for at least 10%, at least 20% at least 50% at least 60%, at least 70%, at least 80%, or at least 90% of the subjects of a given cohort or population. Preferably, treating comprises inhibiting proliferation, more preferably killing, of cancer cells. Preferably, treating cancer is reducing tumor and/or cancer cell burden in a subject. Preferably, treating cancer comprises increasing an immune response to cancer cells of said cancer, more preferably comprises increasing cellular immune response, preferably compared to an untreated control. As will be understood by the skilled person, effectiveness of treatment of e.g. cancer is dependent on a variety of factors including, e.g. cancer stage and cancer type. Also preferably, cancer treatment further comprises at least one of administration of an agent providing a transmembrane E3 ligase, chemotherapy, immunotherapy, surgery, and radiotherapy, wherein immunotherapy preferably comprises administration of T cells, preferably CAR T cells and/or recombinant T cell receptor T cells.

The terms “preventing” and “prevention” refer to retaining health with respect to the diseases or disorders referred to herein for a certain period of time in a subject. It will be understood that the said period of time may be dependent on the amount of the drug compound which has been administered and individual factors of the subject discussed elsewhere in this specification. It is to be understood that prevention may not be effective in all subjects treated with the binding polypeptide. However, the term requires that, preferably, a statistically significant portion of subjects of a cohort or population are effectively prevented from suffering from a disease or disorder referred to herein or its accompanying symptoms. Preferably, a cohort or population of subjects is envisaged in this context which normally, i.e. without preventive measures according to the present invention, would develop a disease or disorder as referred to herein. Whether a portion is statistically significant can be determined without further ado by the person skilled in the art using various well known statistic evaluation tools discussed elsewhere in this specification. In the context of cancer treatment, preventing in particular relates to preventing cancer development, preventing metastasis formation, and/or preventing relapse.

Also, the present invention relates to a method for treating and/or preventing cancer in a subject, said method comprising
(a) administering the binding polypeptide according to the present invention and/or the polynucleotide according to the present invention to said subject, and
(b) thereby treating and/or preventing cancer in said subject.

The aforesaid method of treatment preferably is an in vivo method and may comprise steps in addition to those specifically mentioned. Additional steps may e.g. relate to identifying a subject in need of cancer treatment or at risk of developing cancer, or may relate to further treatment steps as specified herein above.

The present invention also relates to a method for identifying a subject suffering from cancer as being susceptible for treatment with binding polypeptide according to the present invention and/or a polynucleotide according to the present invention, said method comprising
(A) determining expression of a disease-related polypeptide or a surrogate marker thereof in cancer cells of said subject; and
(B) identifying said subject as being susceptible for said treatment based on the determining in step (A).

The method for identifying a subject, preferably, is an in vitro method, and may comprise steps in addition to those specifically indicated. Further steps may e.g. relate to providing a sample, e.g. a cancer sample, for step (A), and/or treating said subject as specified herein above, in particular in case it is determined that the subject is susceptible for said treatment. Whether the subject is susceptible for said treatment can be established by the skilled person based on the indicated determination; i.e., preferably, in case it is determined that cancer cells of said subject express a disease-related polypeptide or of a surrogate marker indicative of disease-related polypeptide expression, the subject preferably is deemed susceptible for said treatment.

In step (A), disease-related polypeptide expression may be determined according to methods known to the skilled person and as shown herein in the Examples, in particular by immunohistochemistry, e.g. on a biopsy sample. However, also a surrogate marker may be determined instead of or in addition to the disease-related polypeptide, the term “surrogate marker” relating to any biological molecule non-identical to the disease-related polypeptide, but indicating its expression in a cell. Such surrogate markers are known in the art and include in particular an mRNA encoding the disease-related polypeptide. Step (A) may comprise additional determinations; preferably, expression of ZNRF3 and/or RNF43 is further determined, and a subject is determined as being susceptible for treatment based in addition on ZNRF3 and/or RNF43 expression. Preferably, in case it is determined that cancer cells of said subject express ZNRF3 and/or RNF43 and, more preferably, ZNRF3 and/or RNF43 do not comprise a loss of function mutation, the subject is deemed susceptible for said treatment.

Also, the present invention relates to a use of a binding polypeptide according to the present invention and/or a polynucleotide according to the present invention for causing degradation of disease-related polypeptide in a host cell and/or for killing a host cell expressing disease-related polypeptide, wherein said use preferably is an in vitro use.

In view of the above, the following embodiments are particularly envisaged:
Embodiment 1: A binding polypeptide comprising a first binding domain binding a transmembrane E3 ligase; and a second binding domain binding a disease-related polypeptide, preferably binding Programmed Death-Ligand 1 (PD-L1).
Embodiment 2: The binding polypeptide of embodiment 1, wherein said first binding domain comprises a furin domain 1 and/or a furin domain 2 of an R-spondin (RSPO), wherein said furin domain 2 is devoid of wnt-activating activity.
Embodiment 3: The binding polypeptide of embodiment 1 or 2, wherein said furin domain 2 comprises an exchange of amino acid F109 for a non-identical amino acid, preferably comprises an F109A amino acid exchange.
Embodiment 4: The binding polypeptide of any one of embodiments 1 to 3, wherein said first binding domain comprises an amino acid sequence at least 80% identical to SEQ ID NO:1.
Embodiment 5: The binding polypeptide of any one of embodiments 1 to 4, wherein said first binding domain comprises furin domain 1 and furin domain 2 of RSPO2.
Embodiment 6: The binding polypeptide of any one of embodiments 1 to 5, wherein said first binding domain comprises an amino acid sequence at least 80% identical to SEQ ID NO:2.
Embodiment 7: The binding polypeptide of any one of embodiments 1 to 6, wherein said first binding domain lacks wnt signaling activity and/or lacks bone morphogenetic protein (BMP) signal inhibiting activity.
Embodiment 8: The binding polypeptide of any one of embodiments 1 to 7, wherein said first binding domain lacks a TSP1 domain, preferably wherein said first binding domain consists of said furin domain 1 and/or said furin domain 2.
Embodiment 9: The binding polypeptide of any one of embodiments 1 to 8, wherein said second binding domain binds the extracellular domain of PD-L1.
Embodiment 10: The binding polypeptide of any one of embodiments 1 to 9, wherein said second binding domain binds an extracellular domain of said disease-related polypeptide.
Embodiment 11: The binding polypeptide of any one of embodiments 1 to 10, wherein said disease-related polypeptide is PD-L1 and wherein said second domain comprises an extracellular IgV-like domain of a Programmed Death-1 (PD-1) polypeptide.
Embodiment 12: The binding polypeptide of any one of embodiments 1 to 11, wherein said second binding domain comprises an amino acid sequence at least 80% identical to SEQ ID NO: 3 or 4.
Embodiment 13: The binding polypeptide of any one of embodiments 1 to 12, wherein said second binding domain comprises an amino acid sequence at least 75% identical to SEQ ID NO: 5.
Embodiment 14: The binding polypeptide of any one of embodiments 1 to 13, wherein said first binding domain and said second binding domain are covalently connected.
Embodiment 15: The binding polypeptide of any one of embodiments 1 to 14, wherein said first binding domain and said second binding domain together form a fusion polypeptide.
Embodiment 16: The binding polypeptide of embodiment 15, wherein said first binding domain and said second binding domain are connected via a linker, preferably a GS linker, more preferably comprising an amino acid sequence of SEQ ID NO:6.
Embodiment 17: The binding polypeptide of any one of embodiments 1 to 16, wherein said binding polypeptide comprises an amino acid sequence at least 80% identical to SEQ ID NO: 7.
Embodiment 18: The binding polypeptide of any one of embodiments 1 to 17, wherein said binding polypeptide consists of an amino acid sequence at least 80% identical to SEQ ID NO: 7.
Embodiment 19: The binding polypeptide of any one of embodiments 1 to 18, wherein said disease-related polypeptide is a transmembrane polypeptide comprising at least one extracellular domain.
Embodiment 20: The binding polypeptide of any one od claims 1 to 19, wherein said disease-related polypeptide is a T cell regulator polypeptide or an immune checkpoint polypeptide.
Embodiment 21: the binding polypeptide of any one of claims 1 to 20, wherein said disease-related polypeptide is PD-L1.
Embodiment 22: A polynucleotide encoding a binding polypeptide according to any one of embodiments 1 to 21.
Embodiment 23: A host cell comprising a binding polypeptide according to any one of embodiments 1 to 21 and/or a polynucleotide according to embodiment 22.
Embodiment 24: A device comprising a binding polypeptide according to any one of embodiments 1 to 21 and/or a polynucleotide according to embodiment 22.
Embodiment 25: A kit comprising a binding polypeptide according to any one of embodiments 1 to 21 and/or a polynucleotide according to embodiment 22, and optionally a means of administration.
Embodiment 26: A method for killing cancer cells, comprising contacting said cancer cells with a binding polypeptide according to any one of embodiments 1 to 21 and/or a polynucleotide according to embodiment 22.
Embodiment 27: The method of embodiment 26, wherein said method is an in vitro method.
Embodiment 28: The method of embodiment 26 or 27, wherein said cancer cell is a PD-L1 overexpressing cancer cell.
Embodiment 29: A binding polypeptide according to any one of embodiments 1 to 21 and/or a polynucleotide according to embodiment 22, for use in medicine.
Embodiment 30: Use of the binding polypeptide according to any one of embodiments 1 to 21 and/or a polynucleotide according to embodiment 22, for the manufacture of a medicament.
Embodiment 31: A binding polypeptide according to any one of embodiments 1 to 21 and/or a polynucleotide according to embodiment 22, for use in treating and/or preventing cancer.
Embodiment 32: The binding polypeptide and/or a polynucleotide for use of embodiment 31, wherein said treating and/or preventing further comprises administration of an agent providing a transmembrane E3 ligase.
Embodiment 33: The binding polypeptide and/or a polynucleotide for use of embodiment 31 or 32, wherein said treating and/or preventing further comprises administration of at least one of immunotherapy, surgery, radiotherapy, and chemotherapy.
Embodiment 34: The binding polypeptide and/or a polynucleotide for use of any one of embodiments 31 to 33, wherein said immunotherapy comprises administration of T cells, preferably CAR T cells and/or recombinant T cell receptor T cells.
Embodiment 35: The binding polypeptide and/or a polynucleotide for use of any one of embodiments 31 to 34, wherein said cancer is melanoma.
Embodiment 36: Use of the binding polypeptide according to any one of embodiments 1 to 21 and/or a polynucleotide according to embodiment 22 for the manufacture of a medicament for treating and/or preventing cancer.
Embodiment 37: A method for treating and/or preventing disease in a subject, said method comprising
(a) administering the binding polypeptide according to any one of embodiments 1 to 22 and/or a polynucleotide according to embodiment 22 to said subject, and
(b) thereby treating and/or preventing cancer in said subject.
Embodiment 38: A method for identifying a subject suffering from cancer as being susceptible for treatment with a binding polypeptide according to any one of embodiments 1 to 21 and/or a polynucleotide according to embodiment 22, said method comprising
(A) determining expression of a disease-related polypeptide, preferably PD-L1 expression, or a surrogate marker thereof in cancer cells of said subject; and
(B) identifying said subject as being susceptible for said treatment based on the determining in step (A).
Embodiment 39: The method of embodiment 38, wherein said subject is identified as being susceptible for said treatment in case it is determined in step (a) that said cancer cells express said disease-related polypeptide, preferably PD-L1, preferably overexpress PD-L1.
Embodiment 40: Use of a binding polypeptide according to any one of embodiments 1 to 21 and/or a polynucleotide according to embodiment 22 for causing degradation of a disease-related polypeptide, preferably PD-L1, in a host cell and/or for killing a host cell expressing a disease-related polypeptide, preferably expressing PD-L1.
Embodiment 41: The subject matter of any one of embodiments 1 to 40, wherein said transmembrane E3 ligase is a ZNRF3 E3 ligase and/or an RNF43 E3 ligase.

All references cited in this specification are herewith incorporated by reference with respect to their entire disclosure content and the disclosure content specifically mentioned in this specification.

### FIGURE LEGENDS

FIG. 1. RSPO chimera bridges ZNRF3/RNF43 and PD-L1. a. Top, domain-structure of indicated proteins. Bottom, structural prediction from RSPO2 (PDB 4ufr) and PD-1 HAC (PDB 5ius). R2^{FA}, RSPO2 furin domains with a LGR binding deficient F¹⁰⁹A mutation. PD1^{HAC}, high-affinity consensus (HAC) mutation of PD-1 with enhanced binding to PD-L1. SP, signal peptide; FU, furin domain; GS, glycine-serine linker; TSP1, thrombospondin domain 1. b. ELISA binding assay. c-e. ELISA assay as in (b) for direct interaction between RSPO chimera and (c) PD-L1^{ECD}, (d) ZNRF3^{ECD}, (e) RNF43^{ECD}. f. ELISA based ternary complex assay. g-h. ELISA based ternary complex assay as in (f). R2PD1 bridges the interaction between PD-L1^{ECD} and (g) ZNRF3BCD and (h) RNF43^{ECD}

FIG. 2. RSPO chimera promotes degradation of overexpressed PD-L1. a. PD-L1 degradation assay. b-e, g. Immunoblot analysis of indicated proteins in 293T cells. Cells were transfected as indicated and analyzed after 24 h incubation with conditioned medium containing the indicated proteins (b, d, g) or indicated purified proteins (c, e). Control conditioned medium and mock-purified protein were used as control. In (c) the normalized ratio between PD-L1 and ERK is indicated. f. Immunofluorescence staining of 293T cells for indicated proteins. Cells were transfected for 2 days with PD-L1-GFP and ZNRF3-HA. After additional 5 h incubation with equal amounts of the indicated purified proteins, cells were fixed and stained for GFP and the lysosomal marker LAMP1. Note PD-L1 colocalization with LAMP1 upon treatment with R2PD1 (arrows). Scale bar, 10 μm. 0.2, 0.8, 2.2, 5.4 and 13.6 nM purified R2PD1 protein used in (c). 5.4 nM purified R2PD1 protein used in (e).

FIG. 3. RSPO chimera promotes degradation of endogenous PD-L1 in cancer cells. a-e, h, j-l, o-p, r, s. Immunoblot analysis of indicated proteins in MEL624 and COLO-800 cells. (a, j) Cells were transfected for 3 days with indicated siRNAs. (b-e, k-l, p, r-s) Cells were analyzed after 24 h incubation or indicated hours with the indicated proteins or conditioned medium. (h) Cells were treated with 333 U IFN-γ for 24 h and harvested for analysis after additional 24 h incubation with purified proteins. (o) Cells without treatment were analyzed. Mock-purified protein or control conditioned medium was used as control. The normalized ratio between PD-L1 and TfR is shown below. f-g, m. Flow cytometry analysis of cell surface PD-L1 in MEL624 (f-g) and COLO-800 (m) cells. Quantification is shown in (g, m). i, n, q. qRT-PCR analysis of the expression of indicated genes in MEL624 and COLO-800 cells. (i, n) Cells were analyzed after 24 h incubation with the indicated purified proteins. (q) Cells without treatment were analyzed. Indicated conditioned medium used in (b). 0.027, 0.081, 0.27, 0.81 and 2.7 nM purified protein used in (c). 0.24 nM purified proteins used in (d). 1 nM purified protein used in (e-g, i). 0.027, 0.081, 0.27, 0.81 and 2.7 nM purified protein used in (h). 0.081, 0.27, 0.81 and 2.7 nM purified protein used in (k). 0.81 nM protein used in (l-n). 0.7 nM purified protein used in (p). (r) 0.07 and 0.24 nM of purified protein or anti-PD-L1 antibody (Atezolizumab), (s) 0.07 and 0.7 nM of purified protein or anti-PD-L1 antibody (Atezolizumab). Mock-purified protein and isotypic antibody control were used as controls.

FIG. 4. PD-L1 degradation by RSPO chimera is independent of WNT/β-cat signaling. a-d. Immunoblot analysis of indicated proteins in MEL624 (a-c) and COLO-800 (d) cells. Cells were analyzed after 12 h (a) or 24 h (b-d) incubation with indicated protein. Cells were transfected with indicated siRNAs for 2 days before incubation with purified protein (c-d). e. qRT-PCR analysis of Axin2 expression in COLO-800 cells. Cells were analyzed after 24 h incubation with purified proteins in presence of WNT3A. f. TOPflash assay with HEK293T cells. Cells were analyzed after 12 h incubation with indicated protein. 10, 50 and 200 ng ml⁻¹ DKK1 protein used in (a-b). 0.24 nM purified R2PD1 protein used in (b). Purified R2PD1 used with 0.24 nM (c), 0.73 nM (d), 0.8 nM (e). 0.12, 0.36, 1.08 and 3.24 nM RSPO2 or R2PD1 in (f).

FIG. 5. PD-L1 degradation by RSPO chimera requires ZNRF3/RNF43. a-g. PD-L1 degradation assay. PD-L1 degradation by R2PD1 in presence of competing soluble RNF43^{ECD}Fc (a-b) or after ZNRF3/RNF43 knock down (c-g). b, d, f, h-i. Immunoblot analysis of indicated proteins in MEL624 (b, d), COLO-800 (f) and A375 (h-i) cells. Cells were analyzed after 24 h incubation with indicated protein. Cells were transfected with indicated siRNAs for 2 days before incubation with purified protein (d, f). e, g. Flow cytometry analysis of cell surface PD-L1 in MEL624 (e) and COLO-800 (g) cells. Values are normalized to the corresponding control group. 50, 150, 500 and 1,500 ng ml⁻¹ RNF43^{ECD}Fc was used in (b). 0.27 nM (b), 0.14 nM (d), 1 nM (e), 0.8 nM (f), 2.5 nM (g) purified R2PD1 used. 0.073, 0.24, 0.73 and 2.44 nM (h-i) purified R2PD1.

FIG. 6: (a) Flow-cytometric quantification of activated cytotoxic CD107a+ T cells. HEK293T cells were transfected for 2 days with plasmids encoding MART-1 minigene, PD-L1 and ZNRF3/RNF43. T cells were transfected for 1 day with indicated mRNAs for TCR and PD-1. After incubation with indicated purified proteins, HEK293T cells were mixed with T cells for 5 h. After 5 h co-incubation, cells were harvested and activated cytotoxic CD107a+CD8+T cells were quantified. 0.4, 1.1, 3.3, 10 and 30 nM purified R2PD1 were used. n=single sample. Representative data from two independent experiments are shown. (b) Flow-cytometric quantification of cytotoxic TNFα+ T cells. T cells were transfected for 1 day with mRNAs expressing TCR and PD-1. MEL624 cells were incubated overnight with the indicated proteins. After additional 5 h co-incubation, MEL624 cells and T cells were analyzed by flow cytometry. Equal numbers of CD8+ T cells were analyzed from each combination. 0.14, 0.41, 1.23, 3.7 and 11.1 nM purified R2PD1 or PD-L1 antibody Atezolizumab was used. n=single sample. Representative data from three independent experiments with the same conclusion are shown. (c-d) Real-time cell growth assay. MEL624 cells were pre-treated with IFNγ for 2 days and mixed with T cells in presence of the indicated proteins. T cells were transfected for 1 day with mRNAs expressing TCR and PD-1. Growth of MEL624 cells was monitored with the XCELLigence system. n=3 experimentally independent samples. Data are normalized to the time point when T cells were added and displayed as mean. Standard error of the mean (SEM) is indicated for the last time point. For (c), **** P<0.0001 from one-way ANOVA test. For (d), **** P<0.0001 from two-tailed paired t-test. 0.41, 3.7 and 11.1 nM purified R2PD1 was used in (c). 0.41 and 3.7 nM purified R2PD1 or 3.7 and 11.1 nM PD-L1 antibody Atezolizumab were used in (d).

The following Examples shall merely illustrate the invention. They shall not be construed, whatsoever, to limit the scope of the invention.

### Example 1: Methods

### 1.1 Cell Lines and Growth Conditions

HEK 293T, MEL624 and A375 cells were maintained in DMEM High glucose (Gibco 11960) supplemented with 10% FBS (Capricorn FBS-12A), 1% penicillin-streptomycin (Sigma P0781), and 2 mM L-glutamine (Sigma G7513). A375 wild type and ZNRF3/RNF43 double knockout cells were gifts from Bryja lab and validated in a previous study (Radaszkiewicz et al., Elife 10, doi: 10.7554/eLife.65759 (2021)). COLO-800 cells were maintained in RPMI (Gibco 21875) with 10% FBS, 1% penicillin-streptomycin, 2 mM L-glutamine and 1 mM sodium pyruvate (Sigma S8636). All cell lines were cultured at 37° C. and 5% CO₂ in a humidity-controlled incubator. Mycoplasma contamination was negative in all cell lines used. The primary Human T cell line was derived from an ex vivo expanded culture of tumor-infiltrating lymphocytes obtained from a pancreatic cancer sample (T222, human, male, pancreatic ductal adenocarcinoma), which was previously described (Poschke et al. (2020), Clin. Cancer Res. 26:4289). To ensure future availability, the cells has been repeatedly expanded using the rapid expansion protocol outlined in the same publication and cryopreserved. Three days before electroporation, cells were thawed and allowed to rest in a 24-well plate containing X-vivo 15 (Lonza BE02-053Q) supplemented with 2% HSA (CSL Behring 01468366) and 300 IU/mL human IL-2 (Clinigen Healthcare 17152.00.00) at a density of 3×106 cells/mL. The cells were maintained in a humidity-controlled incubator at 37° C. and 5% CO2. Mycoplasma contamination tests were negative for all cell lines used.

### 1.2 Constructs

Human RSPO2 wild type construct is C-terminally tagged with Flag in the pCS2+ vector, which was validated in previous studies (Sun et al., Cell rep 36:109559, doi: 10.1016/j.celrep.2021.109559 (2021)). R2^{FA} with the phenylalanine-alanine mutation at position of 109 within FU2 domain was obtained by mutagenesis PCR. PD1^{ECD} was cloned by inserting the N-terminus to the end of IgV-like domain of human PD-1 in pCS2+ vector. The high-affinity consensus mutant of PD-1 (PD1^{HAC}) (Maute et al., Proc Natl Acad Sci USA 112: E6506-6514, doi: 10.1073/pnas. 1519623112 (2015)) was generated by Gibson assembly with synthetic oligos. Human PD-L1 extracellular domain was inserted into the alkaline phosphatase (AP)-pCS2+ vector and expressed as the AP fusion protein (PD-L1^{ECD} AP). RSPO chimera R2^{FA}PD1^{HAC} was obtained through Gibson assembly with fragments from the FU1/2 domains of R2^{FA} and PD1^{HAC} constructs and inserted into pCS2+ vector. A flexible 10-amino acid glycine-serine linker (GSGSGGSGSG, SEQ ID NO:6) was inserted between FU2 domain of RSPO2 and PD1^{HAC} to promote autonomous folding of these domains. Human ZNRF3-HA, ZNRF3^{ΔRING}-myc and RNF43-Flag constructs were described previously (Chang et al., Elife 9, doi:10.7554/eLife.51248 (2020); Kim et al., Elife 10, doi:10.7554/eLife.70885 (2021)). Human PD-L1-GFP, pEGFP-N1/PD-L1 was a gift from Mien-Chie Hung (Addgene plasmid #121478; RRID: Addgene_121478). The sequences of the constructs generated by this study were confirmed with individual DNA sequencing.

1.3 Cell Transfection siRNAs and plasmids were transfected using DharmaFECT 1 transfection reagent (Dharmacon T-2001) and X-tremeGENE9 DNA transfection reagent (Roche 06365809001) respectively, according to the manufacturer protocols.

### 1.4 Generation of Conditioned Medium and Protein Purification

HEK 293T cells were seeded into 10 cm culture dishes and transiently transfected with RSPO2-Flag, R2^{FA}-Flag, PD1-Flag, PD1^{HAC}-Flag, R2PD1-Flag and PD-L1^{ECD}AP. After 24 hours, media were changed to fresh DMEM containing 10% FBS, 1% L-glutamine and 1% penicillin-streptomycin, and harvested daily in the following three days. Conditioned media were validated and quantified by immunoblot. Media containing equal mole amount of proteins were used in the subsequent analysis. WNT3A conditioned medium was produced in L-cells as previously described (Kazanskaya et al., Dev Cel 7:525-534 (2004)). For protein purification, conditioned media were over-night incubated with anti-Flag antibody conjugated agarose beads (Sigma A2220). After washing with icecold PBS, proteins bound to the beads were eluted by 200 mM Glycine (pH 2.6) and neutralized with an equal volume of 1 M Tris (pH 8.0). The eluate was dialyzed against icecold PBS with at least 1000-fold volume excess. Protein concentration was quantified by Coomassie staining using BSA standards (Sigma P0914).

### 1.5 In Vitro Binding Assay

High binding 96-well plates (Greiner M5811) were coated with 2 μg ml⁻¹ of recombinant human PD-L1^{ECD}Fc (Peprotech 310-35), ZNRF3^{ECD}Fc (R&D systems 7994-RF-025) or RNF43^{ECD}Fc (R&D systems 7964-RN-050) protein in bicarbonate coating buffer (50 mM NaHCO₃, pH 9.6) overnight at 4° C. Coated wells were washed three times with TBST (TBS, 0.1% Tween-20) and blocked with 5% BSA in TBST for 1 hour at room temperature. After overnight incubation with Flag tagged proteins, wells were washed three times with TBST. Protein bound to the wells were detected with a peroxidase conjugated anti-Flag antibody (Sigma A8592) and quantified with QuantaBlu™ Fluorogenic Peroxidase Substrate Kit (Thermo Scientific™ 15169). For the ternary complex formation assays, instead of HRP-anti-Flag antibody, PD-L1^{ECD} AP was added to wells. After overnight incubation, AP signal was detected with the chemiluminescent AquaSpark AP substrate (Serva 42593.01). Data are displayed as average of biological replicates with SD.

### 1.6 PD-L1 Degradation Assay

HEK 293T cells were seeded into cell culture plates and transfected with indicated plasmids. 24 hours after transfection, cells were treated with either conditioned media or purified proteins as indicated. After another 24 hours incubation, cells were harvested for immunoblot. Cancer cells were seeded into cell culture plates and treated as indicated. After 24 hours incubation or indicated periods, cells were harvested for immunoblot. For the degradation assay in present of IFNγ stimulation, MEL624 cells were pre-incubated with 333 U IFNγ (ImmunoTools 11343536) for 24 hours. PD-L1 antibody Atezolizumab and matched isotype control used in the assay was obtained from Offringa lab from DKFZ, Germany.

### 1.7 Immunoblot

Cultured cells were harvested and lysed in icecold RIPA buffer with complete Protease Inhibitor Cocktail (Roche 11697498001). Lysates were mixed with Laemmli buffer containing β-mercaptoethanol and boiled at 70° C. for 10 min to prepare SDS-PAGE samples. For cytosolic β-catenin detection, saponin buffer was used. Immunoblot images were acquired with SuperSignal West pico ECL (ThermoFisher 34580) using LAS-3000 system (FujiFilm). Quantification of blots was done using ImageJ software.

### 1.8 Immunofluorescence (IF)

Cells were seeded to cell culture plates with inserted glass coverslips. 24 hours after transfection with indicated plasmids, cells were treated with purified proteins for indicated periods. After fixation with 4% PFA for 10 min, cells were stained with primary antibodies (1:250) overnight at 4° C., and fluorophore conjugated secondary antibodies (1:250) at room temperature for 2 hours. Images were obtained using LSM 700 (Zeiss) and analyzed with ImageJ. Around 100 cells were analyzed in each group. The following primary antibodies were used for staining: chicken anti-GFP (Millipore AB16901), mouse anti-LAMP1 (Cell Signaling Technology 15665).

### 1.9 Quantitative Real-Time PCR

Cultured cells were lysed in Macherey-Nagel RAI buffer containing 1% β-mercaptoethanol and total RNAs were isolated using NucleoSpin RNA isolation kit (Macherey-Nagel 740955). Reverse transcription and PCR amplification were performed as described before (Sun et al., Cell rep 36:109559, doi: 10.1016/j.celrep.2021.109559 (2021)). Primers used for AXIN2 were: forward 5′-CCACACCCTTCTCCAATCC-3′ (SEQ ID NO:15) and reverse 5′-TGCCAGTTTCTTTGGCTCTT-3′ (SEQ ID NO:16). Primers used for PD-L1 were: forward 5′-CCTACTGGCATTTGCTGAACG-3′ (SEQ ID NO:17) and reverse 5′-AGACAATTAGTGCAGCCAGGT-3′ (SEQ ID NO:18). Primers used for ZNRF3 were: forward 5′-TGTGCCATCTGTCTGGAGAA-3′ (SEQ ID NO:21) and reverse 5′-TTCCTGTGAAACCGGTGAGT-3′ (SEQ ID NO:22). Primers used for RNF43 were: forward 5′-GTTTGCTGGTGTTGCTGAAA-3′ (SEQ ID NO:23) and reverse 5′-TGGCATTGCACAGGTACAG-3′ (SEQ ID NO:24). Primers used for GAPDH were: forward 5′-AGCCACATCGCTCAGACAC-3′ (SEQ reverse 5′-ID NO: 19) and GCCCAATACGACCAAATCC-3′ (SEQ ID NO:20). Graphs show relative gene expressions to GAPDH. Data are displayed as mean with SD from multiple experimental replicates.

### 1.10 TOPflash Luciferase Reporter Assay

TOPflash luciferase assays were carried out as previously described (Berger et al., EMBO rep 18:712-725, doi: 10.15252/embr.201643585 (2017)). Data are displayed as average of biological replicates with SD.

### 1.11 Quantification and Statistical Analysis

Statistical analyses were done with the PRISM7 software using unpaired t-test or one-way ANOVA test. Not significant (ns), p>0.05, *p<0.05, ** p<0.01, *** p<0.001, **** p<0.0001.

### 1.12 Flow Cytometry

Cells were harvested through a none-enzymatic approach, pelleted and resuspended in ice-cold blocking buffer (PBS supplemented with 1% BSA and 0.1% NaN3). After blocking with Fc receptor binding inhibitor (eBioscience 14916173), cells were staining with anti-PD-L1 antibody (CST 86744), followed by incubation with a fluorochrome-labeled secondary antibody. Staining with secondary antibody only was used as control. Dead cells were excluded by counterstaining with propidium iodide. FACS Samples were analyzed with FACSCanto or LSRFortessa (BD Biosciences) and data were processed with FlowJo software. Rabbit anti-PD-L1 (CST 86744) antibody was used for cell surface PD-L1 measurement.

For T cell activation assay, intracellular and extracellular stainings were performed using the following antibodies: anti-human CD107a (BioLegend 328608), anti-human TNFa (BioLegend 502915), anti-mouse TRBC (BioLegend 109230), anti-human CD3 (BioLegend 317328), anti-human CD4 (BioLegend 344614), anti-human CD8 (BioLegend 300920) and LIVE/DEAD™ Fixable Aqua Dead Cell Stain Kit (Thermofisher L34966). PD-1 antibody (BioLegend 329906) was used for surface PD-1 expression measurement. Data were acquired on a Fortessa cytometer (BD Biosciences) and analyzed using Flowjo v10.3 software (BD Biosciences).

### 1.13 Co-Culture Assay for Evaluating T Cell Activation

A co-culture assay was carried out to examine the TCR transfected T cell recognition capacity against tumor or other target circumstances. To monitor tumor recognition, CD107a and TNFa levels were measured. A U-bottom 96-well plate with 50,000 effector cells (T cells) and 200,000 target cells was incubated at 37° C. for 5 hours. To accumulate the TNF and CD107a signal in response to T cell activation, commercially available GolgiPlug (BD bioscience 555028) and GolgiStop (BD bioscience 554715) were utilized at 1:000. When human tumor cells (MEL624 and COLO-800) were used as target cells, they were pre-treated for 48 hours with commercially available human IFN-αt 333 U ml⁻¹. TCR reactivity was evaluated by flow cytometry after 5 hours of effector and target cell incubation. Activated T cells percentage were measured by CD107a or TNFa upregulation on viable CD8+ T cells. Transfected effector cells were tested against media, to determine the background reactivity. The result of the test was subtracted to check the reactivity of the effector cells.

### 1.14 Cell Cytotoxicity Assay

The impact of R2PD1 chimera on cell proliferation and cytotoxicity was assessed using the CellTiter-Glo assay (Promega) following the manufacturer's instructions. MEL624 and COLO-800 cells were seeded at a density of 500 cells per well in a 96-well microplate with three wells per group. After an overnight incubation, the R2PD1 chimera were added to the cells in growth medium, and the potential cytotoxicity was measured after 72 hours. CellTiter-Glo reagent (100 μl) was added to each well, and the luminescence was recorded using Spark Multimode Microplate Reader (Tecan). The relative luminescence was obtained by normalizing it as a fold-change relative to the average luminescence of the wells with control medium on the same plate. Protein untreated cells served as a control, and background luminescence was measured in wells containing medium without cells.

1.15 xCELLigence Based Tumor Killing Assay

For tumor killing assay, a 96-well microplate (Agilent 5232376001) with gold microelectrode cell sensors built into the bottom of each well was used together with the xCELLigence system (Agilent). The basal impedance of the medium was measured before adding tumor cells. Each well was seeded with 20,000 tumor cells, and once the tumor cells reached a cell index of one an equal number of effector cells was added. The cell index was measured every minute for the first 30 minutes after the effector cells were added, and then every five minutes thereafter. After starting co-culture, target cell growth was monitored for 4-5 days. Each condition had three independent replicates, which were monitored in parallel. The normalized cell index was calculated by dividing the cell index value at each time point by the cell index at the time of effector cell addition. The RTCA software Pro was used for analysis (Version 2.3.0).

### 2. Results

### 2.1 Design and Validation of a Bi-Specific RSPO Chimera

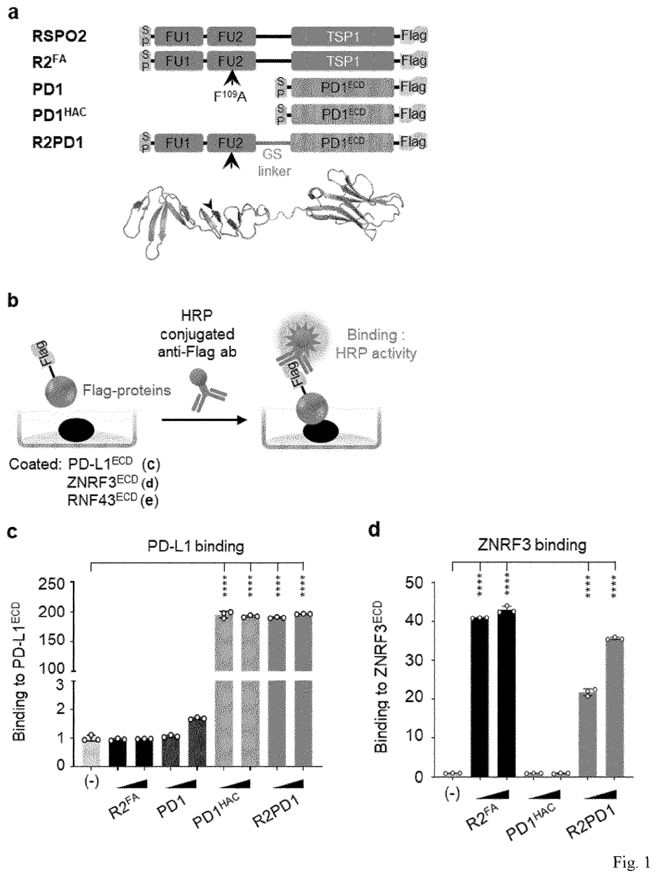

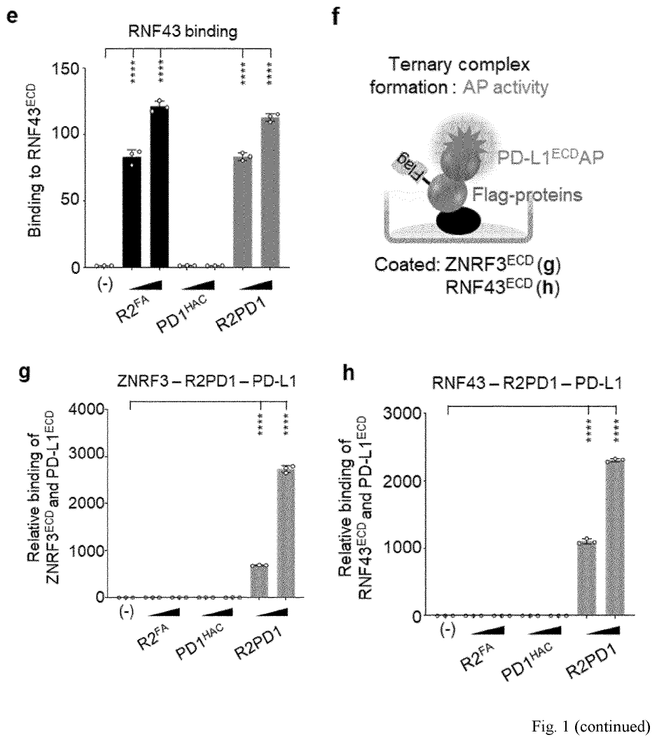

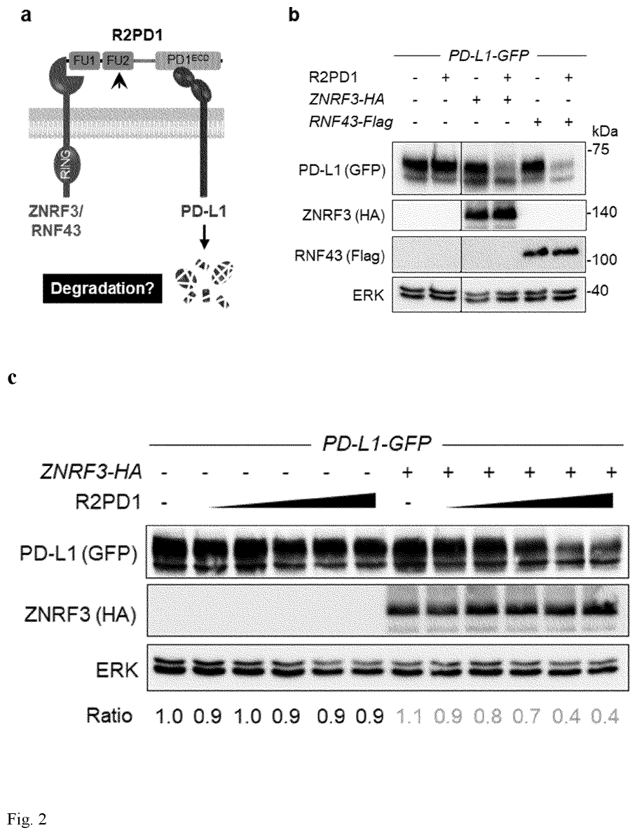

We modified RSPO2, which possesses the highest binding affinity to ZNRF3 (Park et al., J. Biol. Chem. 2018, 293 (25), 9759-9769), to generate a bi-specific chimera with simultaneous binding capacity for ZNRF3/RNF43 and PD-L1 (FIG. 1a). To prevent unwanted WNT signal activation, we mutated phenylalanine 109 of RSPO2 within the FU2 domain (FA mutation), aiming for a LGR-binding deficient mutant (R2^{FA}). To obtain a PD-L1 binding unit, we cloned the extracellular IgV-like domain of PD-1 (PD1) and generated its high-affinity binding variant (Maute et al., Proc Natl Acad Sci USA 2015, 112 (47), E6506-6514) PD1^{HAC}. To obtain the final bi-specific reagent, we fused the RSPO2 FU1/2^{FA} domains with the PD1^{HAC} module via a flexible glycine-serine linker, effectively replacing the TSP1 domain of RSPO2, which is non-essential for ZNRF3/RNF43 binding, resulting in the bi-specific RSPO2 chimera, R2PD1. Replacing the TSP1 domain in RSPO2 also abolishes BMP signaling inhibition.

Recombinant PD1^{HAC}, R2^{FA}, and R2PD1 proteins were readily produced and secreted into the medium of transfected HEK293T cells. Expectedly, R2PD1 bound LGR4 much weaker than the chimera without FA mutation in a cell surface binding assay. To test whether R2PD1 maintains the binding activity towards its interaction partners, we performed solid-phase based ELISA assays (FIG. 1b). Towards immobilized PD-L1, PD1 bound weakly but PD1^{HAC} strongly (FIG. 1c), confirming that HAC mutation conveys higher affinity towards PD-L1. Importantly, R2PD1 and PD1^{HAC} showed equally strong binding towards immobilized PD-L1 (FIG. 1c). Moreover, R2PD1 bound immobilized ZNRF3 and RNF43 similar to R2^{FA} (FIG. 1d-e). Thus, R2PD1 retains tight binding towards its both targets, PD-L1 and ZNRF3/RNF43.

The prerequisite of E3 ligase-mediated protein degradation is the formation of an E3 ligase-target complex. To test if R2PD1 bridges the interaction between ZNRF3/RNF43 and PD-L1, we employed ELISA assays using immobilized ZNRF3 and RNF43 (FIG. 1f). As expected, PD-L1 bound ZNRF3 (FIG. 1g) and RNF43 (FIG. 1h) only in presence of R2PD1, but not of R2^{FA} or isolated PD1^{HAC} protein. We conclude that RSPO chimera R2PD1 effectively bridges the interaction between PD-L1 and ZNRF3/RNF43.

### 2.2 RSPO Chimera Promotes Degradation of Overexpressed PD-L1 293T Cells

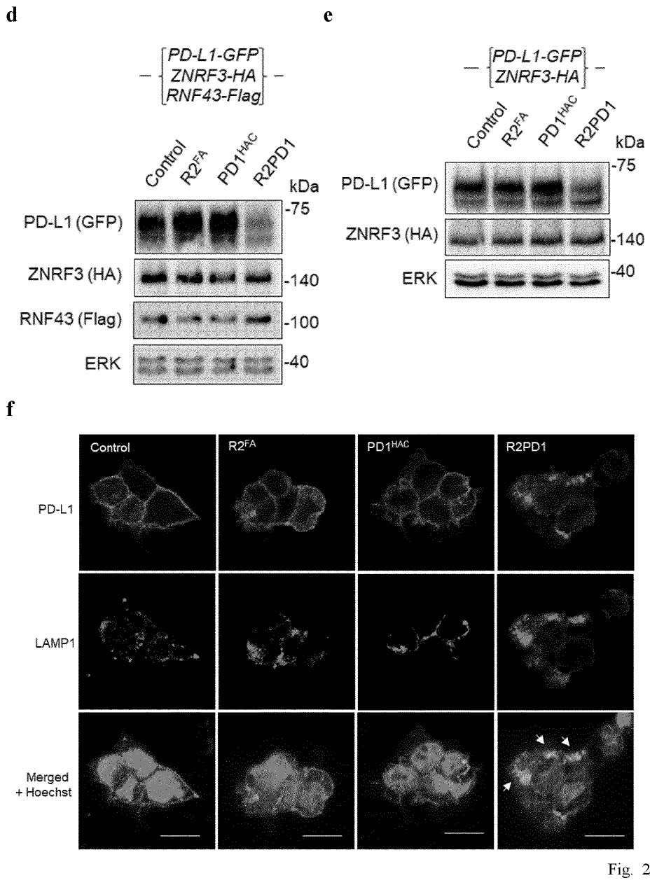

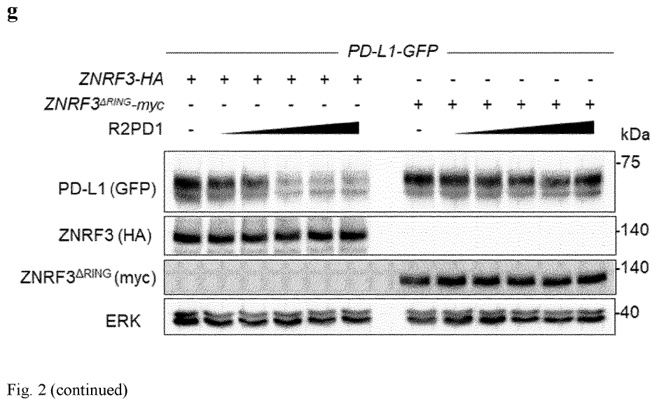

To test whether the RSPO chimera causes degradation of PD-L1, we performed degradation assays utilizing HEK293T cells transfected with human PD-L1 with or without ZNRF3/RNF43 (FIG. 2a). Cotransfection of ZNRF3 or RNF43 had no effect on PD-L1 levels. However, addition of recombinant R2PD1 effectively reduced levels of overexpressed PD-L1, but only when ZNRF3 or RNF43 were contransfected (FIG. 2b). Degradation of PD-L1 reached plateau after incubation with 5.4 nM purified recombinant proteins, with a maximum of 60% total protein reduction detected in an immunoblot (FIG. 2c).

Next, we hypothesized that the degradation of PD-L1 requires simultaneous binding to PD-L1 and ZNRF3/RNF43. Indeed, no reduction of PD-L1 levels was detected in cells incubated with equal amounts of either R2^{FA} or PD1^{HAC} (FIG. 2d-e). This finding was further confirmed by IF, where R2PD1 abolished PD-L1 cell surface staining and instead induced colocalization with the lysosomal marker LAMP1 (FIG. 2f). No such effect was seen with either R2^{FA} or PD1^{HAC} This result is consistent with previous findings that ZNRF3/RNF43 employ the lysosomal pathway for targeted protein degradation.

To test the importance of E3 ligase activity in concert with PD-L1 degradation, we transfected 293T cells with a dominant negative variant of ZNRF3, lacking the intracellular RING domain (ZNRF3^{ΔRING}). R2PD1 efficiently reduced total PD-L1 protein level in a dose-dependent manner in cells expressing full-length ZNRF3, while degradation of PD-L1 was abolished upon ZNRF3^{ΔRING} transfection (FIG. 2g). Collectively, we conclude that RSPO chimera causes degradation of overexpressed PD-L1 in presence of overexpressed ZNRF3/RNF43 in 293T cells.

### 2.3 RSPO Chimera Promotes Degradation of Endogenous PD-L1 in Cancer Cells

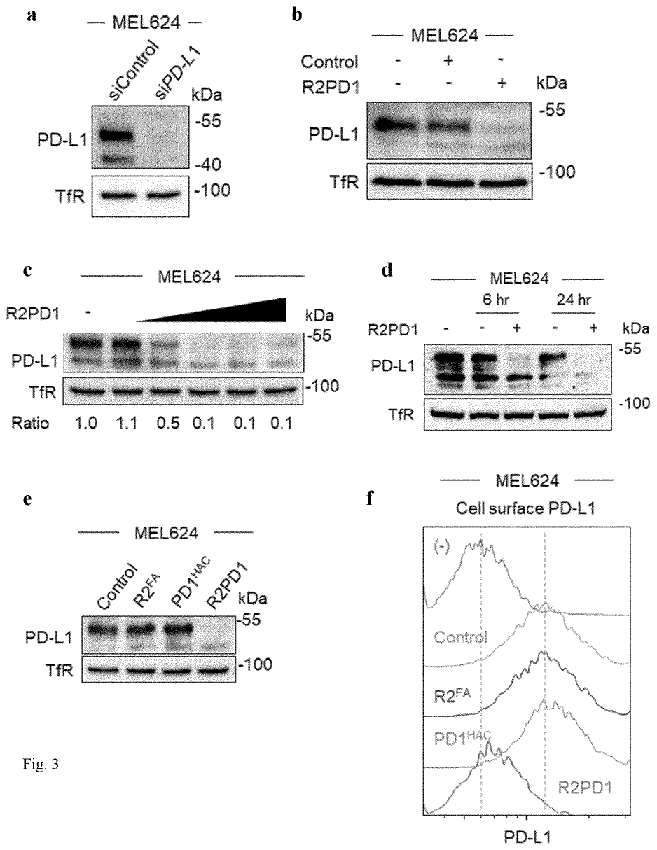

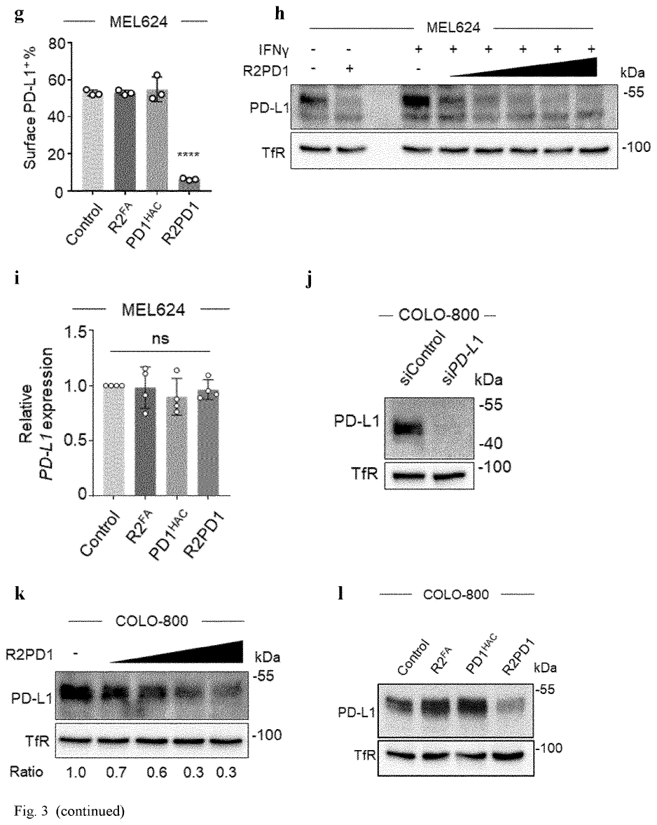

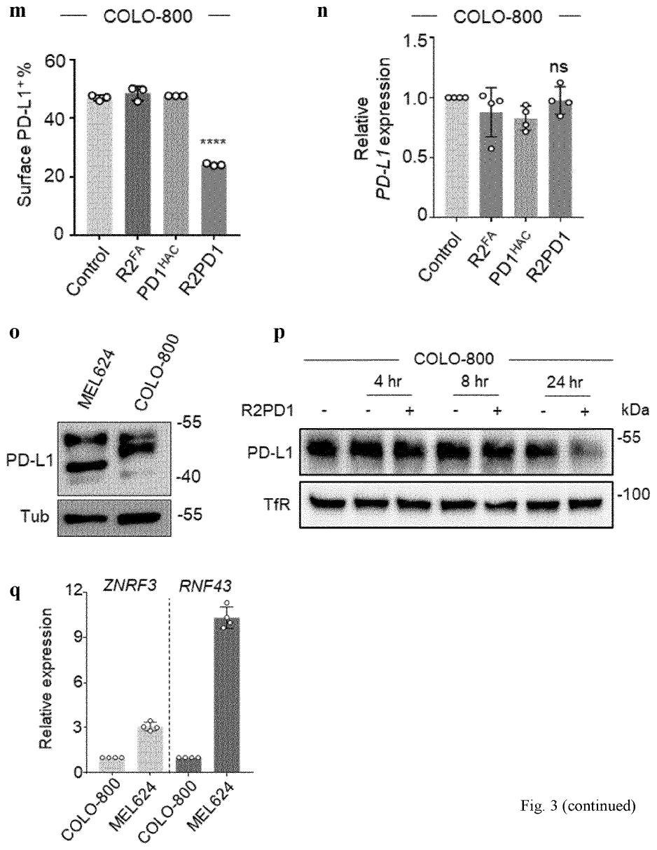

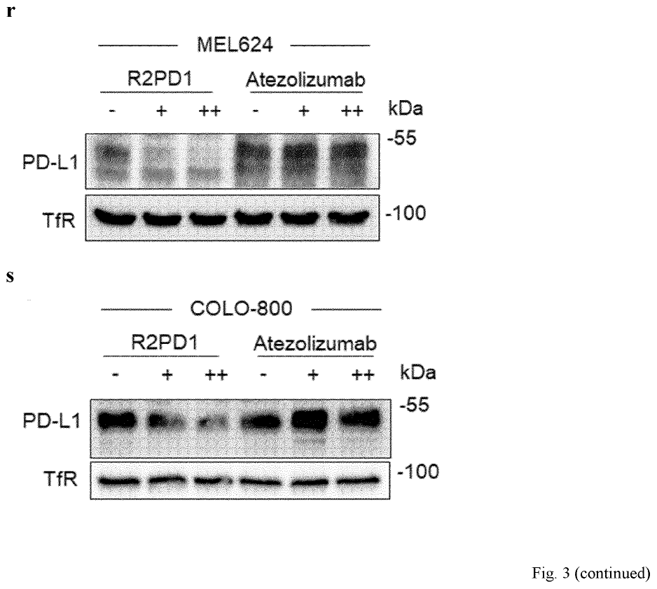

To investigate whether RSPO chimera causes degradation of endogenous PD-L1, we turned to cancer cell lines with PD-L1 expression. PD-L1 small interfering RNA (siRNA) knockdown in the human melanoma cell line MEL624 decreased PD-L1 protein levels, confirming both antibody specificity and that the cells express PD-L1 (FIG. 3a). Addition of R2PD1 conditioned medium reduced PD-L1 level by ~90% (FIG. 3b). PD-L1 reduction plateaued with as little as 0.27 nM R2PD1 protein (FIG. 3c). PD-L1 reduction by R2PD1 was readily detected within 6 hours (FIG. 3d). Moreover, PD-L1 reduction was only observed with R2PD1, but not R2^{FA} or PD1^{HAC} (FIG. 3e), corroborating the requirement for bi-specificity. Flow cytometry analysis confirmed that R2PD1 induced the cell surface removal of PD-L1 (FIG. 3f-g).

The expression of PD-L1 is regulated by various cytokines residing in the tumor microenvironment, notably Interferon-Gamma (IFNγ). We asked whether R2PD1 is capable to degrade PD-L1 induced by IFNγ. Expectedly, IFNγ increased PD-L1 protein level and co-incubation with R2PD1 decreased it (FIG. 3h).

To rule out that reduction of PD-L1 is due to its transcriptional misregulation rather than induced protein degradation, we monitored PD-L1 mRNA in MEL624 cells. qRT-PCR analysis revealed no significant changes of PD-L1 mRNA in cells incubated with the effector proteins under study (FIG. 3i).

We corroborated our finding with another melanoma cell line, COLO-800, where PD-L1 is expressed and can be reduced by siRNA transfection (FIG. 3j). Once again, R2PD1 chimera decreased both the cell surface and the total protein of PD-L1, although less complete than in MEL624 cells (FIG. 3k-m). Again, no significant change of PD-L1 mRNA was detected by qRT-PCR analysis (FIG. 3n). Despite the comparable protein levels of PD-L1 in MEL624 and COLO-800 cells (FIG. 30), the reduction of PD-L1 in COLO-800 was evident only after 24 hours incubation with R2PD1 chimera (FIG. 3p), later than with MEL624 cells (FIG. 3d). We speculated that lower efficiency and distinct kinetics of PD-L1 degradation by R2PD1 is due to the different expression levels of ZNRF3/RNF43. Indeed, qRT-PCR analysis revealed significantly higher expression of ZNRF3/RNF43 in MEL624 cells than those in COLO-800 cells (FIG. 3q).

Atezolizumab is a therapeutic monoclonal antibody targeting PD-L1 and blocking its interaction with PD-1, thereby promoting tumor-directed T-cell activation. Here, we compared the effect of RSPO chimera and Atezolizumab on PD-L1 protein levels. R2PD1 chimera again reduced PD-L1 in both MEL624 and COLO-800 cells, while addition of Atezolizumab did not change PD-L1 protein levels (FIG. 3r-s). Collectively, these data support that R2PD1 chimera causes degradation of endogenous PD-L1 protein in cancer cells.

### 2.4 PD-L1 Degradation by RSPO Chimera is Independent of WNT/B-Cat Signaling

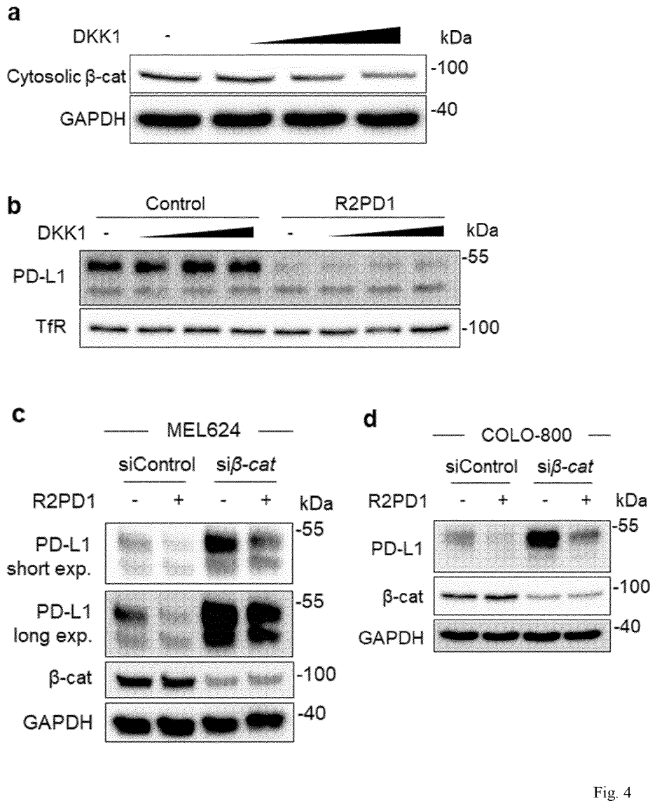

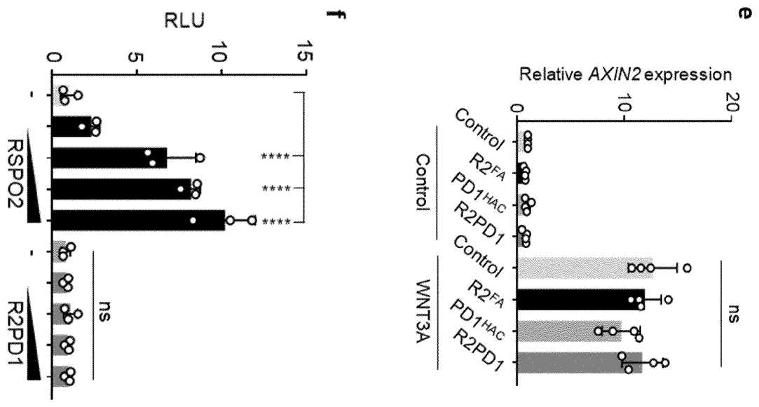

RSPOs are potent WNT signaling agonists and BMP signaling antagonists. Since these signaling activities may lead to unwanted side effects when applying an RSPO chimeric protein, the here used R2PD1 protein is deleted of its TSP1 domain, which completely abolishes its BMP inhibition and it contains a F¹⁰⁹A (FA) mutation within the FU2 domain, that should abrogate LGR-binding and hence should prevent WNT activation. To rule out that the R2PD1 furin domains retain residual WNT/B-cat signaling activity, we performed PD-L1 degradation assays in presence of WNT inhibitors. R2PD1 chimera degraded PD-L1 both in presence of the potent WNT signaling inhibitor DKK1 (FIG. 4a-b) and upon β-catenin siRNA knockdown (FIG. 4c-d). Moreover, the chimeric protein failed to induce expression of the WNT targeted gene AXIN2 or affect WNT reporter (TOPflash) activity, unlike wild-type RSPO2 (FIG. 4e-f). These findings corroborate that the RSPO chimera is WNT signaling-disabled and that PD-L1 degradation is independent of WNT/β-cat signaling.

### 2.5 PD-L1 Degradation by RSPO Chimera Requires ZNRF3/RNF43

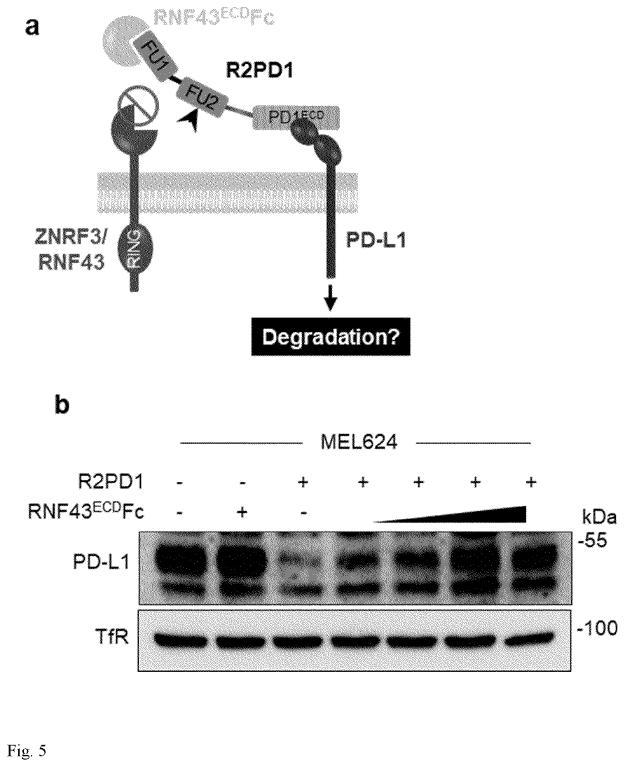

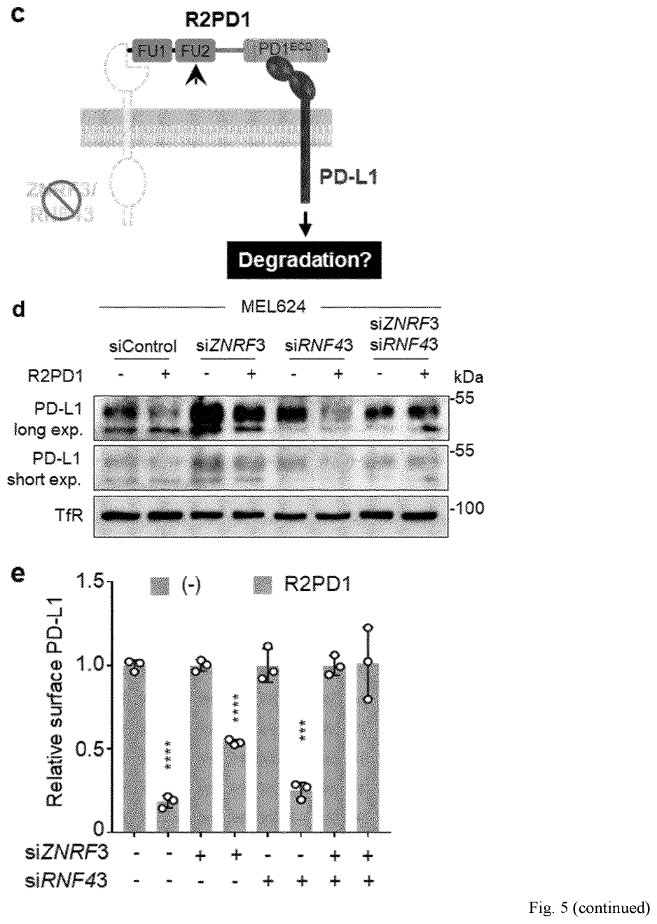

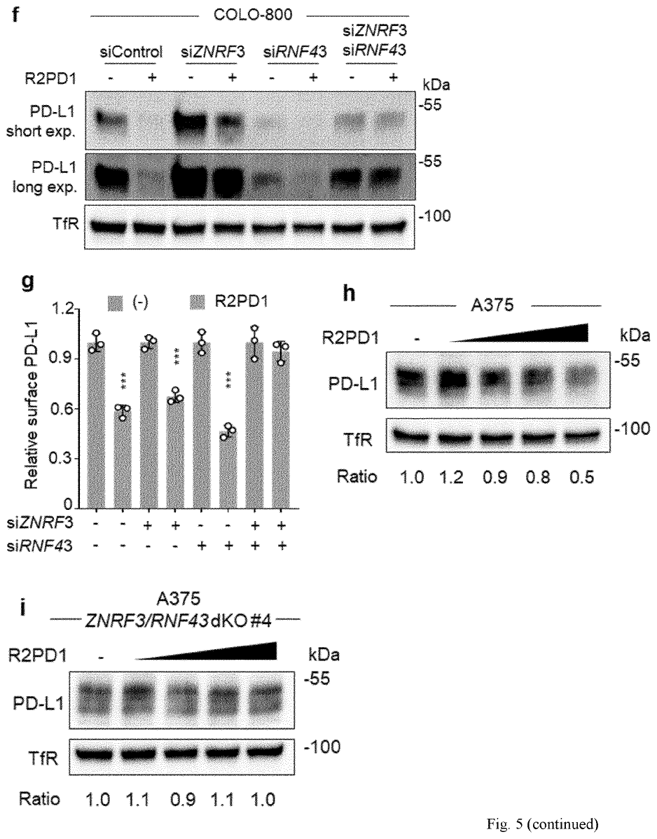

We have shown that the bi-specific chimera R2PD1 causes degradation of PD-L1. To address whether PD-L1 degradation occurs via ZNRF3/RNF43, we incubated R2PD1 chimeric protein with a soluble extracellular domain of RNF43 (RNF43^{ECD}Fc) before adding it to cells (FIG. 5a). The rationale is that since RNF43^{ECD}Fc binds R2PD1 directly (FIG. 1e), it should nullify the effect of R2PD1, provided that the chimera acts specifically. Consistently, R2PD1 pre-incubated with RNF43^{ECD}Fc rescued PD-L1 from degradation (FIG. 5b). Furthermore, while single siRNA knockdown of either ZNRF3 or RNF43 was not sufficient to block the degradation of PD-L1 by R2PD1, double knockdown successfully prevented the degradation in both MEL624 (FIG. 5c-e) and COLO-800 cells (FIG. 5f-g), indicating that the chimera engages both E3 ligases. To corroborate these findings, we utilized a ZNRF3/RNF43 double-mutant A375 melanoma cell line (dKO). While R2PD1 reduced PD-L1 in control A375 cells, it failed to decrease PD-L1 in dKO cells (FIG. 5h-i). We conclude that in melanoma cells, RSPO chimera engages ZNRF3 and RNF43 for PD-L1 degradation.

### 2.6 R2PD1 Chimera Reactivates Cytotoxic T Cells and Inhibits Tumor Cell Proliferation

To validate that targeting PD-L1 by R2PD1 chimera enhances cytotoxic T cell activation, we employed T cell activation- and tumor cell killing assays by co-culturing primary T cells with target cells. Primary T cells were transfected with human DMF5 T cell receptor (TCR) mRNA to enable recognition of target cells presenting the matching MART-1 melanoma antigen. Moreover, to enhance PD-L1/PD-1 signalling, PD-1 mRNA was co-transfected into T cells.

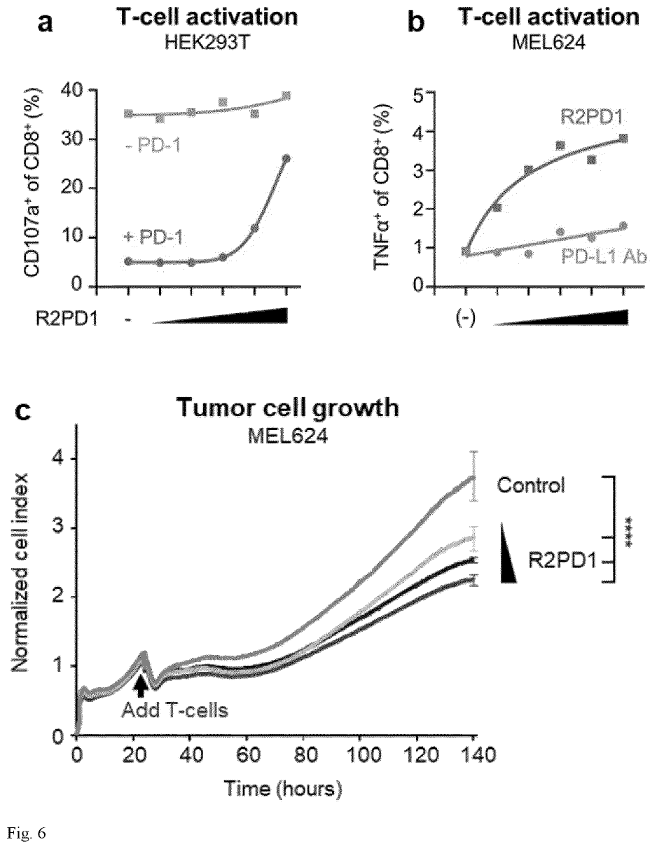

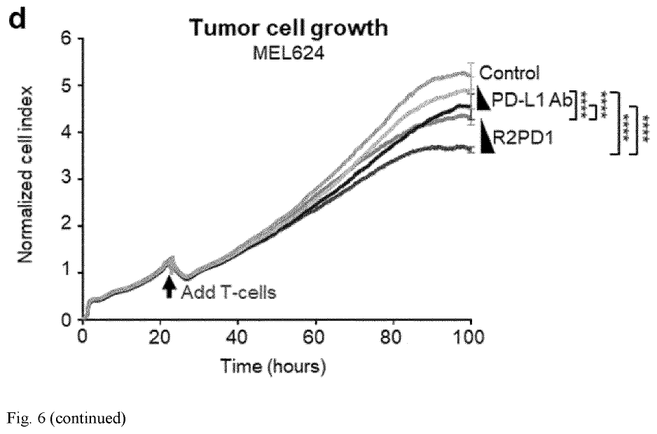

We initially engineered HEK 293T cells as T cell targets by transfecting them with PD-L1 and a MART-1 minigene, and co-cultured them with T cells transfected with a matching TCR. As a marker for cytotoxic T cell activation, we monitored expression of CD107a or TNFα. Upon co-incubation of the transfected cells, ~60% of CD8+ T cells were CD107a-positive. Co-expression of PD-L1/PD-1 in HEK293T cells and T cells, respectively, reduced the percentage of activated T cells by ~80%. When R2PD1 chimera was added to such HEK293T-restricted T cells, it restored T cell activation in a dose-dependent manner as monitored by CD107a expression (FIG. 6 (a)).

Using this experimental regime, we next replaced as T cell-targets engineered HEK293T cells by MEL624 cells, which express MART-1 endogenously. Upon co-cultivation of MEL624 and T cells, R2PD1 chimera activated T cells in a dose-dependent manner (FIG. 6 (b)). When compared to equal concentrations of the clinically used checkpoint inhibitor Atezolizumab, a humanized IgG1 monoclonal antibody that targets PD-L1 we found R2PD1 considerably more potent (FIG. 6 (b)): The R2PD1 response reached saturation at low nM concentrations where Atezolizumab showed no effect yet, and at the highest dose employed, R2PD1 was still 2.5× more potent than Atezolizumab (FIG. 6 (b))

Moreover, R2PD1 chimera reduced growth of MEL624 cells when co-cultivated with T cells (FIG. 6 (c)) but not without T cells. Also in this tumor killing assay, at equal concentration (3.7 nM), R2PD1 was more potent than Atezolizumab, reducing tumor cell growth by 30% vs 7% (FIG. 6 (d)). Note that while overall growth inhibition is moderate in these short-term in vitro assays, in vivo the full tumor-restricting potential of checkpoint inhibitors unfolds over an extended time. We conclude that R2PD1 chimera functions as a checkpoint inhibitor in vitro where it is more potent to reactivate T cells and restrict tumor cell growth than the established checkpoint inhibitor Atezolizumab.

### LITERATURE CITED

Berger et al., EMBO rep 18:712-725, doi: 10.15252/embr.201643585 (2017)
Chang et al., Elife 9, doi: 10.7554/eLife.51248 (2020);
de Lau et al., Genome Biology 13:242 (2012)
de Lau et al., Genes Dev 28:305, doi: 10.1101/gad.235473.113 (2014)
Hao et al., Nature 485:195, doi: 10.1038/nature11019 (2012)
He et al., Front. Cell Dev. Biol 9:685106, doi: 10.3389/fcell.2021.685106 (2021)
Hey et al. (2005), Trends Biotechnol. 23:514
Kazanskaya et al., Dev Cel 7:525-534 (2004)
Kim et al., Elife 10, doi: 10.7554/eLife.70885 (2021)
Koo et al., Nature 488:665, doi: 10.1038/nature11308 (2012)
Lau et al., Genome Biology 13:242 (2012)
Lee et al., Nature comm 11:5570, doi: 10.1038/s41467-020-19373-w (2020)
Maute et al., Proc Natl Acad Sci USA 112: E6506-6514, doi: 10.1073/pnas. 1519623112 (2015)
Park et al., J. Biol. Chem. 2018, 293 (25), 9759-9769)
Poschke et al. (2020), Clin. Cancer Res. 26:4289
Radaszkiewicz et al., Elife 10, doi: 10.7554/eLife.65759 (2021)
Sun et al, Signal Transduct Target Ther 4:64 doi: 10.1038/s41392-019-0101-6 (2019)
Sun et al., Cell rep 36:109559, doi: 10.1016/j.celrep.2021.109559 (2021)
ter Stege et al. (2021), Oncogene 40 (47): 6469
Zebisch et al., Nature comm 4:2787, doi: 10.1038/ncomms3787 (2013)

1-17. (canceled)

18. A binding polypeptide comprising a first binding domain binding a transmembrane E3 ligase; and a second binding domain binding a disease-related polypeptide,
wherein said first binding domain comprises a furin domain 1 and/or a furin domain 2 of an R-spondin (RSPO), and
wherein said first binding domain lacks wnt signaling activity and/or lacks bone morphogenetic protein (BMP) signal inhibiting activity.

19. The binding polypeptide claim 18, wherein said first binding domain comprises furin domain 1 and furin domain 2 of RSPO2.

20. The binding polypeptide claim 19, wherein said furin domain 2 comprises an exchange of amino acid F109 for a non-identical amino acid, preferably comprises an F109A amino acid exchange.

21. The binding polypeptide of claim 18, wherein said first binding domain comprises an amino acid sequence at least 80% identical to SEQ ID NO:2.

22. The binding polypeptide of claim 18, wherein said first binding domain lacks a TSP1 domain.

23. The binding polypeptide of claim 18, wherein said first binding domain consists of said furin domain 1 and/or said furin domain 2.

24. The binding polypeptide of claim 18, wherein said disease-related polypeptide is a T cell regulator polypeptide or an immune checkpoint polypeptide.

25. The binding polypeptide of claim 18, wherein said disease-related polypeptide is Programmed Death-Ligand 1 (PD-L1), and wherein said second binding domain comprises an extracellular IgV-like domain of a Programmed Death-1 (PD-1) polypeptide.

26. The binding polypeptide of claim 18, wherein said second binding domain comprises an amino acid sequence at least 80% identical to SEQ ID NO:3 or 4, preferably wherein said second binding domain comprises an amino acid sequence at least 75% identical to SEQ ID NO:5.

27. The binding polypeptide of claim 18, wherein said first binding domain and said second binding domain together form a fusion polypeptide, preferably wherein said first binding domain and said second binding domain are connected via a linker, preferably a GS linker, more preferably comprising an amino acid sequence of SEQ ID NO:6.

28. The binding polypeptide of claim 18, wherein said binding polypeptide comprises an amino acid sequence at least 80% identical to SEQ ID NO:7.

29. A polynucleotide encoding a binding polypeptide according to claim 18.

30. A method for killing cancer cells, comprising contacting said cancer cells with a binding polypeptide according to claim 18.

31. The method of claim 30, wherein said cancer cell is a PD-L1 overexpressing cancer cell.

32. A method of treating and/or preventing cancer comprising
(a) administering the binding polypeptide according to claim 18 to said subject, and
(b) thereby treating and/or preventing cancer in said subject.

33. The method of claim 32, wherein said treating and/or preventing further comprises administration of an agent providing a transmembrane E3 ligase.

34. The method of claim 32, wherein said treating and/or preventing further comprises administration of at least one of immunotherapy, surgery, radiotherapy, and chemotherapy.

35. The method of claim 34, wherein said immunotherapy comprises administration of T cells, preferably CAR T cells and/or recombinant T cell receptor T cells.

36. The method of claim 32, wherein said cancer is melanoma.

37. A method for identifying a subject suffering from cancer as being susceptible for treatment with a binding polypeptide according to claim 18, said method comprising:
(A) determining PD-L1 expression or a surrogate marker thereof in cancer cells of said subject; and
(B) identifying said subject as being susceptible for said treatment based on the determining in step (A).
